SEQUENCE LISTING (42 sequences)

SEQ ID NO: 1 - RNA, 23 nt, synthetic construct
  source 1..23: mol_type = other RNA
ttacaaagagtctgtaataatgg

SEQ ID NO: 2 - RNA, 23 nt, synthetic construct
  source 1..23: mol_type = other RNA
taatgtgggatttccccagttgg

SEQ ID NO: 3 - RNA, 23 nt, synthetic construct
  source 1..23: mol_type = other RNA
ttccccagttggagtaagggtgg

SEQ ID NO: 4 - RNA, 23 nt, synthetic construct
  source 1..23: mol_type = other RNA
atagccacccttactccaactgg

SEQ ID NO: 5 - RNA, 23 nt, synthetic construct
  source 1..23: mol_type = other RNA
aaaaaagaatggaccttgtgtgg

SEQ ID NO: 6 - RNA, 23 nt, synthetic construct
  source 1..23: mol_type = other RNA
tgtggtcactgccggatggtagg

SEQ ID NO: 7 - RNA, 23 nt, synthetic construct
  source 1..23: mol_type = other RNA
tcaggagtggccaccgcgctcgg

SEQ ID NO: 8 - RNA, 23 nt, synthetic construct
  source 1..23: mol_type = other RNA
acacgttgtgagattttaagagg

SEQ ID NO: 9 - RNA, 23 nt, synthetic construct
  source 1..23: mol_type = other RNA
agcttacagacccggttctcagg

SEQ ID NO: 10 - RNA, 23 nt, synthetic construct
  source 1..23: mol_type = other RNA
ggctcagaggtgaagcactgtgg

SEQ ID NO: 11 - RNA, 23 nt, synthetic construct
  source 1..23: mol_type = other RNA
attcggcgaagtggcggagctgg

SEQ ID NO: 12 - RNA, 23 nt, synthetic construct
  source 1..23: mol_type = other RNA
gtgcccgcgcctccgcaccgcgg

SEQ ID NO: 13 - RNA, 23 nt, synthetic construct
  source 1..23: mol_type = other RNA
aaagccgaaggcggagcagctgg

SEQ ID NO: 14 - RNA, 23 nt, synthetic construct
  source 1..23: mol_type = other RNA
tttgcccactctcttcgcagtgg

SEQ ID NO: 15 - RNA, 23 nt, synthetic construct
  source 1..23: mol_type = other RNA
tcctgggtccccaccgggcacgg

SEQ ID NO: 16 - RNA, 23 nt, synthetic construct
  source 1..23: mol_type = other RNA
ggcggggctccttccccagcagg

SEQ ID NO: 17 - RNA, 23 nt, synthetic construct
  source 1..23: mol_type = other RNA
agcgaatgcgactggcggggcgg

SEQ ID NO: 18 - RNA, 23 nt, synthetic construct
  source 1..23: mol_type = other RNA
gagctgccgccatggccccgcgg

SEQ ID NO: 19 - RNA, 23 nt, synthetic construct
  source 1..23: mol_type = other RNA
tttccttcttgcagcctgtgcgg

SEQ ID NO: 20 - RNA, 23 nt, synthetic construct
  source 1..23: mol_type = other RNA
gctgggcggctactctagaccgg

SEQ ID NO: 21 - RNA, 23 nt, synthetic construct
  source 1..23: mol_type = other RNA
tccctttcctcaggaagacttgg

SEQ ID NO: 22 - RNA, 23 nt, synthetic construct
  source 1..23: mol_type = other RNA
ggatgcgcgccagcccagcccgg

SEQ ID NO: 23 - RNA, 23 nt, synthetic construct
  source 1..23: mol_type = other RNA
gaagcttccgcagctgaacctgg

SEQ ID NO: 24 - RNA, 23 nt, synthetic construct
  source 1..23: mol_type = other RNA
cgggtctccaggttcagctgcgg

SEQ ID NO: 25 - RNA, 23 nt, synthetic construct
  source 1..23: mol_type = other RNA
cccaagcgacgggcaccatgtgg

SEQ ID NO: 26 - RNA, 23 nt, synthetic construct
  source 1..23: mol_type = other RNA
agctgcggaagcttcccacatgg

SEQ ID NO: 27 - RNA, 23 nt, synthetic construct
  source 1..23: mol_type = other RNA
cccacatggtgcccgtcgcttgg

SEQ ID NO: 28 - RNA, 23 nt, synthetic construct
  source 1..23: mol_type = other RNA
cctgcaggaacaccagccaccgg

SEQ ID NO: 29 - RNA, 23 nt, synthetic construct
  source 1..23: mol_type = other RNA
tagaggaggagaccggtggctgg

SEQ ID NO: 30 - RNA, 23 nt, synthetic construct
  source 1..23: mol_type = other RNA
cggtggctggtgttcctgcaggg

SEQ ID NO: 31 - RNA, 23 nt, synthetic construct
  source 1..23: mol_type = other RNA
gggcaagcgcaaggacatcgagg

SEQ ID NO: 32 - RNA, 23 nt, synthetic construct
  source 1..23: mol_type = other RNA
aagttctgcgccccaggccccgg

SEQ ID NO: 33 - RNA, 23 nt, synthetic construct
  source 1..23: mol_type = other RNA
gccccggcagggacggaaacagg

SEQ ID NO: 34 - RNA, 23 nt, synthetic construct
  source 1..23: mol_type = other RNA
aaacacctcccgcggagactggg

SEQ ID NO: 35 - DNA, 69 bp, synthetic construct
  source 1..69: mol_type = other DNA
gtccttgcgcttgccctgcaagttctgcgccccaggccccggcagggacggaaacaggccgggaagcag

SEQ ID NO: 36 - DNA, 69 bp, synthetic construct
  source 1..69: mol_type = other DNA
gtccttgcgctcgccctgcaggttctgcgccccaggacccggcaaggacggaaacaggccgggaagtag

SEQ ID NO: 37 - DNA, 36 bp, synthetic construct
  source 1..36: mol_type = other DNA
aggcaggtgcccgcgcctccgcaccgcggcgacacc

SEQ ID NO: 38 - DNA, 36 bp, synthetic construct
  source 1..36: mol_type = other DNA
aggcaggtgccagcgcctccgcaccgcggcgacacc

SEQ ID NO: 39 - DNA, 36 bp, synthetic construct
  source 1..36: mol_type = other DNA
gtaaggtcctgggtccccaccgggcacggcccatgc

SEQ ID NO: 40 - DNA, 36 bp, synthetic construct
  source 1..36: mol_type = other DNA
gtaaggtccttggtccccaccgagcacggcccatgc

SEQ ID NO: 41 - DNA, 35 bp, synthetic construct
  source 1..35: mol_type = other DNA
agcgaaagcgaatgcgactggcggggcggcaggtc

SEQ ID NO: 42 - DNA, 35 bp, synthetic construct
  source 1..35: mol_type = other DNA
agcgaaagcgactgcgcctggcggggccgcaggtc

(19) United States
(12) Patent Application Publication
(10) Pub. No.: US 2025/0186615 A1
(43) Pub. Date: Jun. 12, 2025

(54) Induction of IL-15/IL-15RA Expression in Immune Cells

(71) Applicant: Temple University-Of The Commonwealth System of Higher Education, Philadelphia, PA (US)

(72) Inventor: RAFAL KAMINSKI, Philadelphia, PA (US)

(21) Appl. No.: 18/978,810

(22) Filed: Dec. 12, 2024

Related U.S. Application Data
(60) Provisional application No. 63/608,924, filed on Dec. 12, 2023.

(51) Int. Cl. A61K 48/00; A61K 38/17; A61K 38/20; A61P 31/18; A61P 37/04; C12N 9/22; C12N 15/11

(52) U.S. Cl. CPC A61K 48/005; A61K 38/1793; A61K 38/2086; A61P 31/18; A61P 37/04; C12N 9/22; C12N 15/111; C07K 2319/71; C12N 2310/20

(57) ABSTRACT

Described herein are systems and methods for increasing expression of IL-15Ra and IL-15 in immune cells to improve their proliferation and cytolytic functions and method of use to treat or prevent diseases or disorders.

### CROSS REFERENCE TO RELATED APPLICATIONS

This application claims priority to U.S. Provisional Application No. 63/608,924, filed Dec. 12, 2023, which is hereby incorporated by reference herein in its entirety.

### STATEMENT REGARDING FEDERALLY SPONSORED RESEARCH OR DEVELOPMENT

This invention was made with government support under UM1 AI164568 awarded by the National Institutes of Health. The government has certain rights in the invention.

### REFERENCE TO SEQUENCE LISTING SUBMITTED ELECTRONICALLY

This application contains a Sequence Listing, which is submitted electronically via EFS-Web as an XML Document formatted sequence listing with a file name “206017-0259-00US Sequence Listing.xml,” having a creation date of Dec. 11, 2024, and having a size of 38,396 bytes. The sequence listing submitted via EFS-Web is part of the specification and is herein incorporated by reference in its entirety.

### BACKGROUND OF THE INVENTION

Functional restoration of HIV-specific cytotoxic T lymphocytes (CTLs) and natural killer (NK) cells is required for cytotoxic clearance of residual HIV-infected cells. CTLs and NKs mediate strong suppression of HIV infection. Chronic HIV-1 infection drives functional exhaustion of antigen-specific CTLs and NK cells. HIV-specific CTLs and NK cells are functionally impaired with poor cytolytic capacity which cannot be restored by ART. Functional restoration of broad CTL and NK cell response is needed to clear latent HIV infection.

Thus, there is a need in the art for a method of increasing the ability of CTLs to clear latent HIV infection.

### SUMMARY OF THE INVENTION

The present invention relates generally to compositions and methods for increasing the expression or activity of IL-15Ra on immune cells and increasing IL-15. In some embodiments, the invention provides a combination of a) an IL-15Ra activator and b) IL-15, a nucleic acid molecule encoding IL-15, or an activator of IL-15.

In one embodiment, the invention relates to a genome editing system comprising a targeted transcriptional activator comprising a catalytically dead CRISPR Cas protein linked to a transcription activation domain and at least one sgRNA molecule comprising nucleotide sequence that is complementary to the promoter of IL-15Ra. In one embodiment, the invention relates to a genome editing system comprising a targeted transcriptional activator comprising a catalytically dead CRISPR Cas protein linked to a transcription activation domain at least one sgRNA molecule comprising nucleotide sequence that is complementary to the promoter of IL-15.

In one embodiment, the at least one sgRNA molecule comprising nucleotide sequence that is complementary to the promoter of IL-15Ra is one of SEQ ID NO: 1-18.

In one embodiment, the at least one sgRNA molecule comprising nucleotide sequence that is complementary to the promoter of IL-15 is selected from the group consisting of SEQ ID NO: 19-34.

In one embodiment, the system comprises an mRNA molecule encoding the catalytically dead CRISPR Cas protein linked to a transcription activation domain.

In one embodiment, the transcription activation domain comprises a fusion protein comprising VP16, p65 and Rta.

In one embodiment, the system comprises a combination of: a) at least one sgRNA molecule comprising nucleotide sequence that is complementary to the promoter of IL-15Ra, and b) at least one sgRNA molecule comprising nucleotide sequence that is complementary to the promoter of IL-15.

In one embodiment, the invention relates to a method of increasing the expression of the combination of IL-15Ra and IL-15 in a subject, comprising administering to the subject a genome editing system comprising a targeted transcriptional activator comprising a catalytically dead CRISPR Cas protein linked to a transcription activation domain, at least one sgRNA molecule comprising nucleotide sequence that is complementary to the promoter of IL-15Ra, and at least one sgRNA molecule comprising nucleotide sequence that is complementary to the promoter of IL-15.

In one embodiment, the method comprises administering a first composition comprising: a genome editing system comprising a targeted transcriptional activator comprising a catalytically dead CRISPR Cas protein linked to a transcription activation domain, and at least one sgRNA molecule comprising nucleotide sequence that is complementary to the promoter of IL-15Ra, and a second composition comprising a genome editing system comprising a targeted transcriptional activator comprising a catalytically dead CRISPR Cas protein linked to a transcription activation domain, and at least one sgRNA molecule comprising nucleotide sequence that is complementary to the promoter of IL-15.

In one embodiment, the method comprises administering a first composition comprising: a genome editing system comprising an mRNA molecule encoding a targeted transcriptional activator comprising a catalytically dead CRISPR Cas protein linked to a transcription activation domain, and at least one sgRNA molecule comprising nucleotide sequence that is complementary to the promoter of IL-15Ra, and a second composition comprising a genome editing system comprising an mRNA molecule encoding a targeted transcriptional activator comprising a catalytically dead CRISPR Cas protein linked to a transcription activation domain, and at least one sgRNA molecule comprising nucleotide sequence that is complementary to the promoter of IL-15.

In one embodiment, the invention relates to a method of increasing the proliferation of an immune cell in a subject, comprising administering to the subject a genome editing system comprising a targeted transcriptional activator comprising a catalytically dead CRISPR Cas protein linked to a transcription activation domain, at least one sgRNA molecule comprising nucleotide sequence that is complementary to the promoter of IL-15Ra, and at least one sgRNA molecule comprising nucleotide sequence that is complementary to the promoter of IL-15.

In one embodiment, the method comprises administering a first composition comprising: a genome editing system comprising a targeted transcriptional activator comprising a catalytically dead CRISPR Cas protein linked to a transcription activation domain, and at least one sgRNA molecule comprising nucleotide sequence that is complementary to the promoter of IL-15Ra, and a second composition comprising a genome editing system comprising a targeted transcriptional activator comprising a catalytically dead CRISPR Cas protein linked to a transcription activation domain, and at least one sgRNA molecule comprising nucleotide sequence that is complementary to the promoter of IL-15.

In one embodiment, the method comprises administering a first composition comprising: a genome editing system comprising an mRNA molecule encoding a targeted transcriptional activator comprising a catalytically dead CRISPR Cas protein linked to a transcription activation domain, and at least one sgRNA molecule comprising nucleotide sequence that is complementary to the promoter of IL-15Ra, and a second composition comprising a genome editing system comprising an mRNA molecule encoding a targeted transcriptional activator comprising a catalytically dead CRISPR Cas protein linked to a transcription activation domain, and at least one sgRNA molecule comprising nucleotide sequence that is complementary to the promoter of IL-15.

In one embodiment, the immune cell is a T cell, natural killer (NK) cell, myeloid cell, antigen presenting cell, dendritic cell, macrophage, or B cell.

In one embodiment, the immune cell is a cytotoxic T lymphocyte or an NK cell.

In one embodiment, the invention relates to a method of increasing the cytotoxicity of an immune cell in a subject, comprising administering to the subject a genome editing system comprising a targeted transcriptional activator comprising a catalytically dead CRISPR Cas protein linked to a transcription activation domain, at least one sgRNA molecule comprising nucleotide sequence that is complementary to the promoter of IL-15Ra, and at least one sgRNA molecule comprising nucleotide sequence that is complementary to the promoter of IL-15.

In one embodiment, the method comprises administering a first composition comprising: a genome editing system comprising a targeted transcriptional activator comprising a catalytically dead CRISPR Cas protein linked to a transcription activation domain, and at least one sgRNA molecule comprising nucleotide sequence that is complementary to the promoter of IL-15Ra, and a second composition comprising a genome editing system comprising a targeted transcriptional activator comprising a catalytically dead CRISPR Cas protein linked to a transcription activation domain, and at least one sgRNA molecule comprising nucleotide sequence that is complementary to the promoter of IL-15.

In one embodiment, the method comprises administering a first composition comprising: a genome editing system comprising an mRNA molecule encoding a targeted transcriptional activator comprising a catalytically dead CRISPR Cas protein linked to a transcription activation domain, and at least one sgRNA molecule comprising nucleotide sequence that is complementary to the promoter of IL-15Ra, and a second composition comprising a genome editing system comprising an mRNA molecule encoding a targeted transcriptional activator comprising a catalytically dead CRISPR Cas protein linked to a transcription activation domain, and at least one sgRNA molecule comprising nucleotide sequence that is complementary to the promoter of IL-15.

In one embodiment, the immune cell is a cytotoxic T lymphocyte or an NK cell.

In one embodiment, the invention relates to a method of treating or preventing a disease or disorder in a subject, comprising administering to the subject a genome editing system comprising a targeted transcriptional activator comprising a catalytically dead CRISPR Cas protein linked to a transcription activation domain, at least one sgRNA molecule comprising nucleotide sequence that is complementary to the promoter of IL-15Ra, and at least one sgRNA molecule comprising nucleotide sequence that is complementary to the promoter of IL-15.

In one embodiment, the method comprises administering a first composition comprising: a genome editing system comprising a targeted transcriptional activator comprising a catalytically dead CRISPR Cas protein linked to a transcription activation domain, and at least one sgRNA molecule comprising nucleotide sequence that is complementary to the promoter of IL-15Ra, and a second composition comprising a genome editing system comprising a targeted transcriptional activator comprising a catalytically dead CRISPR Cas protein linked to a transcription activation domain, and at least one sgRNA molecule comprising nucleotide sequence that is complementary to the promoter of IL-15.

In one embodiment, the method comprises administering a first composition comprising: a genome editing system comprising an mRNA molecule encoding a targeted transcriptional activator comprising a catalytically dead CRISPR Cas protein linked to a transcription activation domain, and at least one sgRNA molecule comprising nucleotide sequence that is complementary to the promoter of IL-15Ra, and a second composition comprising a genome editing system comprising an mRNA molecule encoding a targeted transcriptional activator comprising a catalytically dead CRISPR Cas protein linked to a transcription activation domain, and at least one sgRNA molecule comprising nucleotide sequence that is complementary to the promoter of IL-15.

In one embodiment, the disease or disorder is cancer or an infectious disease or disorder.

In one embodiment, the disease or disorder is HIV infection or AIDS.

### BRIEF DESCRIPTION OF THE DRAWINGS

The following detailed description of various embodiments of the invention will be better understood when read in conjunction with the appended drawings. For the purpose of illustrating the invention, illustrative embodiments are shown in the drawings. It should be understood, however, that the invention is not limited to the precise arrangements and instrumentalities of the embodiments shown in the drawings.

FIG. 1 depicts a schematic of IL-15Ra/IL-15 signaling in cytotoxic immune cells activation.

FIG. 2A and FIG. 2B depict representative data demonstrating CRISPR-Cas9 gRNA screen for human IL-15Ra gene promoter. FIG. 2A depicts a schematic of human IL-15Ra gene structure. Exons (coding sequence) are shown as white rectangles, transcription start sites (TSS), transcription termination sites (TTS), start codon (ATG), and stop codon (TGA) are shown. The positions of CRISPR gRNAs binding sites in the promoter are shown as arrows.

FIG. 2B depicts representative results of RT-qPCR. K562-dCas9-VPR cells (stably expressing dCas9-VPR protein, purchased from Horizon) were electroporated with candidate CRISPR gRNAs. Three days later cells were harvested, RNA was extracted and used in RT-qPCRs using primers specific to human IL-15Ra and GAPDH as a reference.

FIG. 3A and FIG. 3B depict representative data demonstrating CRISPR-Cas9 gRNA screen for human IL-15 gene promoter. FIG. 3A depicts a schematic of IL-15 gene structure. Exons (coding sequence) are shown as white rectangles, transcription start sites (TSS), transcription termination sites (TTS), start codon (ATG), and stop codon (TGA) are shown. The positions of CRISPR gRNA binding sites in the promoter are shown as arrows. FIG. 3B depicts representative results of RT-qPCR. K562-dCas9-VPR cells (stably expressing dCas9-VPR protein, purchased from Horizon) were electroporated with candidate CRISPR gRNAs. Three days later cells were harvested, RNA was extracted and used in RT-qPCRs using primers specific to human IL-15 and GAPDH as a reference.

FIG. 4 depicts representative data validating lead CRISPR gRNAs targeting IL-15Ra gene promoter in primary human CD8+ T cells. Bar graphs depict representative results of RT-qPCR. Primary human CD8+ T cells were isolated from the blood of three different healthy donors and electroporated with dCas9-VPR mRNA together with lead CRISPR gRNAs targeting human IL-15Ra gene promoter. Three days later cells were harvested, RNA was extracted and used in RT-qPCRs using primers specific to human IL-15Ra and GAPDH as a reference.

FIG. 5 depicts representative data validating lead CRISPR gRNAs targeting IL-15 gene promoter in primary human CD8+ and CD4+ T cells. Bar graphs depict representative results of RT-qPCR. Primary human CD8+ and CD4+ T cells were isolated from the blood of three different healthy donors and electroporated with dCas9-VPR mRNA together with lead CRISPR gRNAs targeting human IL-15 gene promoter. Three days later cells were harvested, RNA was extracted and used in RT-qPCRs using primers specific to human IL-15 and GAPDH as a reference.

FIG. 6 depicts representative data validating lead CRISPR gRNAs targeting IL-15Ra gene promoter in NK cells. Bar graphs depict representative results of RT-qPCR. Primary human NK cells were isolated from the blood of two different healthy donors and electroporated with dCas9-VPR mRNA together with lead CRISPR gRNAs targeting human IL-15Ra gene promoter. Every day for three days cells were harvested, RNA was extracted and used in RT-qPCRs using primers specific to human IL-15Ra and GAPDH as a reference.

FIG. 7 depicts representative data validating lead CRISPR gRNAs targeting IL-15Ra gene promoter in primary human NK cells. Images depict representative results of immunolabeling/flow cytometry. Primary human NK cells were isolated from the blood of two different healthy donors and electroporated with dCas9-VPR mRNA together with lead CRISPR gRNAs targeting human IL-15Ra gene promoter. Every day for three days cells were immunolabeled with anti-IL-15Ra-PE antibody and analyzed for surface expression of IL-15Ra.

FIG. 8 depicts a diagram outlining the hypothesis that NK persistence and function is enhanced by dCas9-mediated induction of IL-15/IL-15Ra expression.

FIG. 9 depicts representative data demonstrating concomitant dCas9-VPR/gRNA-mediated induction of IL-15 and IL-15Ra expression leads to increased proliferation and survival of primary human NK cells in vitro. Primary human NK cells were isolated from the blood of healthy donor using EasySep CD56 positive selection kit (STEMCELL Technologies) and expanded for two weeks in ImmunoCult medium. Next, cells were electroporated with mRNA for dCas9-VPR (Horizon Biosciences) and non-targeting gRNAs (NT), IL-15 promoter targeting gRNAs, IL-15Ra promoter targeting gRNAs or the mix of both IL-15+IL-15Ra promoters targeting gRNAs. 24 h later cells were counted and plated at the density of 10⁶ cells/ml in RPMI medium supplemented with 10% FBS without or with 1 ng/ml or 10 ng/ml of recombinant IL-15 or in complete ImmunoCult medium. After three days the microscopic pictures of the culture wells were taken (left) and cells were stained with viability dye Propidium Iodide and live cells (PI-negative) were counted on Guava MiniCyte (right). The number of live cells was highest in the wells containing cells treated with the mix of both IL-15+IL-15Ra promoters targeting gRNAs (white bars) compared cells treated with non-targeting gRNAs (NT, black bars) or treated with gRNAs targeting IL-15 promoter only (dark grey bars) or IL-15Ra promoter only (light grey bars). Although this increase of viable cell numbers was observed in all culture conditions, the most robust effects were observed in the absence or suboptimal concentration (1 ng/ml) of IL-15 in the growth medium.

FIG. 10A and FIG. 10B depict concomitant dCas9-VPR/gRNA-mediated induction of IL-15 and IL-15Ra expression leads to increased metabolic activity of primary human NK cells in vitro. Primary human NK cells treated with dCas9-VPR and non-targeting (blue-Control) or the mix of IL-15+IL-15Ra promoters targeting gRNAs (green-Experimental) and cultured for three days without (FIG. 10A) or with suboptimal concentration of IL-15 (1 ng/ml, FIG. 10B) were used NK cell metabolic profiling assay (Agilent Seahorse XFp). Cells treated with the mix of both IL-15+IL-15Ra promoters targeting gRNAs (green-Experimental) show higher metabolic activity (glycolytic and mitochondrial activity) compared to cells treated with non-targeting gRNAs (blue-Control). The effects were more pronounced in the absence of IL-15 in the growth medium (FIG. 10A). OCR stands for oxygen consumption rate, which is proportional to mitochondrial respiration. ECAR stands for extracellular acidification rate and measures mitochondrial oxidative phosphorylation based on the OCR, through real-time and live cell analysis.

FIG. 11 depicts representative data demonstrating concomitant dCas9-VPR/gRNA-mediated induction of IL-15 and IL-15Ra expression leads to increased cytolytic activity of primary human NK cells in vitro. Primary human NK cells treated with non-targeting gRNAs (NT), IL-15 promoter targeting gRNAs, IL-15Ra promoter targeting gRNAs or the mix of both IL-15+IL-15Ra promoters targeting gRNAs and cultured for three days without (top) or with a suboptimal concentration of IL-15 (1 ng/ml, bottom) were used in degranulation (left) and cytotoxic killing assays (right). For the degranulation assay NK cells were incubated with anti-CD107a-FITC antibody (a marker of degranulation=cytolytic granule release) and FarRed stained K562 target cells in the ratio NK:K562 5:1 for 1 h and then incubated with Monensin (to prevent quenched fluorescence) for 4 h and analyzed for CD107a-FITC positivity using Guava MiniCyte. As shown in A, the number of CD107a positive NK cells (=releasing cytolytic granules) was the highest in the wells containing cells treated with the mix of both IL-15+IL-15Ra promoters targeting gRNAs (white bars) compared cells treated with non-targeting gRNAs (NT, black bars) or treated with gRNAs targeting IL-15 promoter only or IL-15Ra promoter only (grey bars). In parallel, the same NK cells were incubated with isogenic HIV-1-GFP infected CD4+ T target cells in the ratio NK:CD4+ T cells 5:1. After four hours cells were collected, fixed with 2% paraformaldehyde and analyzed for GFP positivity using Guava MiniCyte. As shown in B, the greater decrease of GFP positive (=HIV-1-infected) CD4+ T cells was observed in wells containing NK cells treated with IL-15, IL-15Ra (grey bars) and both IL-15+IL-15Ra (white bars) promoters targeting gRNAs compared to wells with NK cells treated with non-targeting gRNAs (black bars).

FIG. 12 shows an example of the Primary NK Cell Experimental Workflow.

FIG. 13 depicts the validation of lead CRISPR gRNAs targeting IL-15Ra gene promoter in primary human NK cells. Results of immunolabeling/flow cytometry and RT-qPCR. Primary human NK cells were isolated from the blood of two different healthy donors and electroporated with dCas9-VPR mRNA together with lead CRISPR gRNAs targeting human IL-15Ra gene promoter. Every day for three days cells were immunolabeled with anti-IL-15Ra-PE antibody and analyzed for surface expression of IL-15Ra as well as had RNA extracted for RT-qPCR.

FIG. 14 depicts a summary of the data in FIG. 13.

FIG. 15 demonstrates the induction of IL-15/IL-15Ra expression in primary human NK cells. Immunolabeling/flow cytometry (IL-15Ra only) (bottom left) and RTqPCR data (top) collected from NK cells isolated from 6 different blood donors. All data points collected 24 h after electroporation. RNA levels (evaluated with TaqMan probes) for both IL-15 and IL-15Ra were significantly elevated in cells receiving IL-15+IL-15Ra targeting gRNAs, showing proof of concept for the multiplex induction of the two genes from the same treatment in primary NK cells.

FIG. 16 demonstrates the induction of IL-15 and IL-15Ra expression in macaque NK cells, showing that human equivalent gRNAs targeting IL-15 and IL-15Ra promoters are functional in primary macaque NK cells.

### DESCRIPTION OF THE INVENTION

In one aspect, the invention relates to a genome editing system comprising mRNA for dCas9-VPR, at least one gRNA molecule comprising a targeting domain that is complementary with a target sequence of an IL-15RA gene, and at least one gRNA molecule comprising a second targeting domain that is complementary with a target sequence of an IL-15 gene.

In one embodiment, the at least one gRNA molecule comprising a targeting domain that is complementary with a target sequence of an IL-15RA gene is one of SEQ ID NO: 1-18.

In one embodiment, the at least one gRNA molecule comprising a targeting domain that is complementary with a target sequence of an IL-15 gene is one of SEQ ID NO: 19-34.

In one embodiment, the present invention relates to a method of increasing the expression of an IL-15RA and an IL-15 gene in a cell, comprising administering to the cell a genome editing system comprising mRNA for dCas9-VPR, at least one gRNA molecule comprising a targeting domain that is complementary with a target sequence of an IL-15RA gene, and at least one gRNA molecule comprising a second targeting domain that is complementary with a target sequence of an IL-15 gene.

In one embodiment, the present invention relates to a method of treating or preventing HIV infection or AIDS in a subject, comprising administering to the subject a genome editing system comprising mRNA for dCas9-VPR, at least one gRNA molecule comprising a targeting domain that is complementary with a target sequence of an IL-15RA gene, and at least one gRNA molecule comprising a second targeting domain that is complementary with a target sequence of an IL-15 gene.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +20%, +10%, +5%, +1%, or +0.1% from the specified value, as such variations are appropriate to perform the disclosed methods.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The term “activate,” as used herein, means to induce or increase an activity or function, for example, about ten percent relative to a control value. Preferably, the activity is induced or increased by 50% compared to a control value, more preferably by 75%, and even more preferably by 95%. “Activate,” as used herein, also means to increase a molecule, a reaction, an interaction, a gene, an mRNA, and/or a protein's expression, stability, function or activity by a measurable amount or to increase entirely. Activators are compounds that, e.g., bind to, partially or totally induce stimulation, increase, promote, induce activation, activate, sensitize, or up regulate a protein, a gene, and an mRNA stability, expression, function and activity, e.g., agonists.

“Antisense” refers particularly to the nucleic acid sequence of the non-coding strand of a double stranded DNA molecule, or to a sequence which is substantially homologous to the non-coding strand. As defined herein, an antisense sequence is complementary to the sequence of a double stranded DNA molecule. It is not necessary that the antisense sequence be complementary solely to the coding portion of the coding strand of the DNA molecule. The antisense sequence may be complementary to regulatory sequences specified on the coding strand of a DNA molecule, which regulatory sequences control expression of the coding sequences.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

An “effective amount” or “therapeutically effective amount” of a compound is that amount of a compound which is sufficient to provide a beneficial effect to the subject to which the compound is administered. An “effective amount” of a delivery vehicle is that amount sufficient to effectively bind or deliver a compound.

The term “inhibit,” as used herein, means to suppress or block an activity or function, for example, about ten percent relative to a control value. Preferably, the activity is suppressed or blocked by 50% compared to a control value, more preferably by 75%, and even more preferably by 95%. “Inhibit,” as used herein, also means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

The terms “patient,” “subject,” “individual,” and the like are used interchangeably herein, and refer to any animal, or cells thereof whether in vitro or in vivo, amenable to the methods described herein. In certain non-limiting embodiments, the patient, subject or individual is a human.

A “therapeutic” treatment is a treatment administered to a subject who exhibits signs or symptoms of a disease or disorder, for the purpose of diminishing or eliminating those signs or symptoms.

As used herein, “treating a disease or disorder” means reducing the severity and/or frequency with which a sign or symptom of the disease or disorder is experienced by a patient.

The phrase “biological sample” as used herein, is intended to include any sample comprising a cell, a tissue, or a bodily fluid in which expression of a nucleic acid or polypeptide is present or can be detected. Samples that are liquid in nature are referred to herein as “bodily fluids.” Biological samples may be obtained from a patient by a variety of techniques including, for example, by scraping or swabbing an area of the subject or by using a needle to obtain bodily fluids. Methods for collecting various body samples are well known in the art.

As used herein, an “immunoassay” refers to any binding assay that uses an antibody capable of binding specifically to a target molecule to detect and quantify the target molecule.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific.

In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

A “coding region” of a gene consists of the nucleotide residues of the coding strand of the gene and the nucleotides of the non-coding strand of the gene.

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

“Isolated” means altered or removed from the natural state. For example, a nucleic acid or a peptide naturally present in its normal context in a living animal is not “isolated,” but the same nucleic acid or peptide partially or completely separated from the coexisting materials of its natural context is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, “conjugated” refers to covalent attachment of one molecule to a second molecule.

“Variant” as the term is used herein, is a nucleic acid sequence or an amino acid sequence that differs in sequence from a reference nucleic acid sequence or amino acid sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis. In some embodiments, the variant sequence is at least about 85%, 86%, 87%, 88%, 89%, 90%, 91%, 92%, 93%, 94%, 95%, 96%, 97%, 98%, or 99%, identical to the reference sequence.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates generally to compositions and methods for increasing the expression or activity of IL-15Ra on immune cells and increasing IL-15. In some embodiments, the invention provides a combination of a) an IL-15Ra activator and b) IL-15, a nucleic acid molecule encoding IL-15, or an activator of IL-15. In some embodiments, the methods enhance an immune response against an infectious disease (e.g., a bacterial or viral infection.) In one embodiment, the invention provides compositions and methods for the treatment or prevention of HIV infection or AIDS.

### Targeted Transcriptional Activator

In one embodiment, the invention relates to a composition comprising a targeted transcriptional activator for targeted activation of IL-15Ra, IL-15, or a combination thereof. In one embodiment, the targeted transcriptional activator comprises targeting domain linked to a transcriptional activation domain. Exemplary transcription activation domains include, but are not limited to, VP16, VP64, VPR (VP16 or VP64, p65 and Rta), TET, SunTag, Synergistic Activation Mediator (SAM), CREB binding protein (CBP), and SphI postoctamer homology (SPH). In some embodiments, the transcriptional activation domain comprises VP16, p65 and Rta. In some embodiments, the transcriptional activation domain comprises VP64, p65 and Rta.

In some embodiments, the targeting domain targets the transcriptional activator to a specific genomic location. In some embodiments, the targeting domain comprises a catalytically dead CRISPR Cas protein. Non-limiting examples of Cas proteins include Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2. Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, SpCas9, StCas9, NmCas9, SaCas9, CjCas9, CjCas9, AsCpf1, LbCpf1, FnCpf1, VRER SpCas9, VQR SpCas9, xCas9 3.7, homologs thereof, orthologs thereof, or modified versions thereof. In one embodiment, Cas protein is catalytically deficient (dCas).

In one embodiment, the Cas protein has DNA binding activity. In one embodiment, Cas protein is dCas9.

In one embodiment, the transcriptional activator comprises a dCas9-VP16-p65-Rta fusion. In one embodiment, the transcriptional activator comprises a dCas9

CRISPR Single-Guide RNAs (sgRNAs)

In one aspect, the invention provides targeting nucleic acids, including a CRISPR single-guide RNA (sgRNA) for targeting the transcriptional activator to a target nucleic acid sequence. In one embodiment, the targeting nucleic acid is a targeting RNA oligo. In one embodiment, the RNA oligo comprises a guide sequence comprises a sequence having sufficient complementarity to the target sequence. In one embodiment, the targeting RNA oligo can be delivered in combination with the transcriptional activator to direct the transcriptional activator to a specific target genomic locus.

In one embodiment, the guide RNA is from 15 to 35 nt. In one embodiment, the guide RNA is at least 15 nucleotides. In one embodiment the guide RNA is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer.

In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. Preferably the guide sequence is 10 30 nucleotides long. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

In some embodiments of CRISPR-Cas systems, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and advantageously tracr RNA is 30 or 50 nucleotides in length. However, an aspect of the invention is to reduce off-target interactions, e.g., reduce the guide interacting with a target sequence having low complementarity. Indeed, in the examples, it is shown that the invention involves mutations that result in the CRISPR-Cas system being able to distinguish between target and off-target sequences that have greater than 80% to about 95% complementarity, e.g., 83%-84% or 88-89% or 94-95% complementarity (for instance, distinguishing between a target having 18 nucleotides from an off-target of 18 nucleotides having 1, 2 or 3 mismatches). Accordingly, in the context of the present invention the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

IL-15Ra and IL-15 sgRNA

In one aspect, the nucleic acid molecules of the disclosure comprise a targeted transcriptional activator and a sgRNA targeted to the promotor to drive the expression of IL-15Ra or IL-15. In one embodiment, the targeted transcriptional activator is capable of driving expression of IL-15Ra or IL-15 in an immune cell. Examples of immune cells that can be engineered for increased expression or secretion of IL-15Ra or IL-15 include, but are not limited to, T cells (including killer T cells, helper T cells, regulatory T cells, and gamma delta T cells), natural killer (NK) cells, myeloid cells, antigen presenting cells, dendritic cells, macrophages, and B cells.

In one embodiment, the sgRNA targeted to the IL-15 promotor comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs: 1-18.

In one embodiment, the sgRNA targeted to the IL-15Ra promotor comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs: 19-34.

### Nucleic Acids

The isolated nucleic acid sequences of the disclosure can be obtained using any of the many recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The nucleic acid molecules of the present invention can be modified to improve stability in serum or in growth medium for cell cultures. Modifications can be added to enhance stability, functionality, and/or specificity and to minimize immunostimulatory properties of the nucleic acid molecule of the invention. For example, in order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect function of the molecule.

In one embodiment of the present invention the nucleic acid molecule may contain at least one modified nucleotide analogue. For example, the ends may be stabilized by incorporating modified nucleotide analogues.

Non-limiting examples of nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In exemplary backbone-modified ribonucleotides the phosphoester group connecting to adjacent ribonucleotides is replaced by a modified group, e.g., of phosphothioate group. In exemplary sugar-modified ribonucleotides, the 2′ OH-group is replaced by a group selected from H, OR, R, halo, SH, SR, NH2, NHR, NR2 or ON, wherein R is C1-C6 alkyl, alkenyl or alkynyl and halo is F, Cl, Br or I.

Other examples of modifications are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino) propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications may be combined.

In some instances, the nucleic acid molecule comprises at least one of the following chemical modifications: 2′-H, 2′-O-methyl, or 2′-OH modification of one or more nucleotides. In certain embodiments, a nucleic acid molecule of the invention can have enhanced resistance to nucleases. For increased nuclease resistance, a nucleic acid molecule, can include, for example, 2′-modified ribose units and/or phosphorothioate linkages. For example, the 2′ hydroxyl group (OH) can be modified or replaced with a number of different “oxy” or “deoxy” substituents. For increased nuclease resistance the nucleic acid molecules of the invention can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleobase modifications such as 2-amino-A, 2-thio (e.g., 2-thio-U), G-clamp modifications, can also increase binding affinity to a target.

In one embodiment, the nucleic acid molecule includes a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In one embodiment, the nucleic acid molecule includes at least one 2′-O-methyl-modified nucleotide, and in some embodiments, all of the nucleotides of the nucleic acid molecule include a 2′-O-methyl modification.

Nucleic acid agents discussed herein include otherwise unmodified RNA and DNA as well as RNA and DNA that have been modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, or as occur naturally in the human body. The art has referred to rare or unusual, but naturally occurring, RNAs as modified RNAs, see, e.g., Limbach et al. (Nucleic Acids Res., 1994, 22:2183-2196). Such rare or unusual RNAs, often termed modified RNAs, are typically the result of a post-transcriptional modification and are within the term unmodified RNA as used herein. Modified RNA, as used herein, refers to a molecule in which one or more of the components of the nucleic acid, namely sugars, bases, and phosphate moieties, are different from that which occur in nature, or different from that which occurs in the human body. While they are referred to as “modified RNAs” they will of course, because of the modification, include molecules that are not, strictly speaking, RNAs. Nucleoside surrogates are molecules in which the ribophosphate backbone is replaced with a non-ribophosphate construct that allows the bases to be presented in the correct spatial relationship such that hybridization is substantially similar to what is seen with a ribophosphate backbone, e.g., non-charged mimics of the ribophosphate backbone.

Modifications of the nucleic acid of the invention may be present at one or more of, a phosphate group, a sugar group, backbone, N-terminus, C-terminus, or nucleobase.

The present invention also includes a vector in which the isolated nucleic acid of the present invention is inserted. The art is replete with suitable vectors that are useful in the present invention.

In brief summary, the expression of natural or synthetic nucleic acids encoding a transcriptional activator of the disclosure is typically achieved by operably linking a nucleic acid encoding the transcriptional activator of the disclosure or portions thereof to a promoter, and incorporating the construct into an expression vector. The vectors to be used are suitable for replication and, optionally, integration in eukaryotic cells. Typical vectors contain transcription and translation terminators, initiation sequences, and promoters useful for regulation of the expression of the desired nucleic acid sequence.

The vectors of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The isolated nucleic acid of the invention can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the vector may be provided to a cell in the form of a viral vector. Viral vector technology is well known in the art and is described, for example, in Sambrook et al. (2012, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome or lipid nanoparticle. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

### Nanoparticle Formulations

In one embodiment, the composition of the invention may comprise a nanoparticle, including but not limited to a lipid nanoparticle (LNP), comprising a transcriptional activator, a sgRNA, or a combination thereof, of the invention, or a LNP comprising a nucleic acid encoding a transcriptional activator, a sgRNA, or a combination thereof, of the invention. In some embodiments, the composition comprises an mRNA molecule that encodes all or part of a a transcriptional activator of the invention.

In one embodiment, the immunogenic composition of the invention may comprise a composition comprising a combination of NA antibodies of the invention, or a LNP comprising one or more nucleic acid molecules encoding a combination of NA antibodies of the invention. In one embodiment, the immunogenic composition of the invention may comprise a composition comprising a combination of LNP, wherein the combination of LNP comprises one or more nucleic acid molecules encoding a combination of NA antibodies of the invention.

In one embodiment, the LNP comprises or encapsulates an mRNA molecule encoding a transcriptional activator and at least one sgRNA of SEQ ID NO:1-18, or a fragment or variant thereof.

In one embodiment, the LNP comprises or encapsulates an mRNA molecule encoding a transcriptional activator and at least one sgRNA of SEQ ID NO:19-34, or a fragment or variant thereof.

In one embodiment, the invention provides a combination of LNPs comprising a first LNP comprising or encapsulating an mRNA molecule encoding a transcriptional activator and at least one sgRNA of SEQ ID NO: 1-18, or a fragment or variant thereof and a second LNP comprising or encapsulating an mRNA molecule encoding a transcriptional activator and at least one sgRNA of SEQ ID NO: 19-34, or a fragment or variant thereof.

In one embodiment, the composition further comprises one or more additional immunostimulatory agents. Immunostimulatory agents include, but are not limited to, an additional antigen or antigen binding molecule, an immunomodulator, or an adjuvant.

### Excipients and Other Components of the Composition

The composition may further comprise a pharmaceutically acceptable excipient. The pharmaceutically acceptable excipient can be functional molecules such as vehicles, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and the poly-L-glutamate may be present in the composition at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the composition. The composition may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the vaccine is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The composition may further comprise a genetic facilitator agent as described in U.S. Ser. No. 021,579 filed Apr. 1, 1994, which is fully incorporated by reference.

The composition can be formulated according to the mode of administration to be used. An injectable pharmaceutical composition can be sterile, pyrogen free and particulate free. An isotonic formulation or solution can be used. Additives for isotonicity can include sodium chloride, dextrose, mannitol, sorbitol, and lactose. The composition can comprise a vasoconstriction agent. The isotonic solutions can include phosphate buffered saline. The composition can further comprise stabilizers including gelatin and albumin. The stabilizers can allow the formulation to be stable at room or ambient temperature for extended periods of time, including LGS or polycations or polyanions.

### Treatment Methods

In one embodiment, the invention provides a method for treatment or prevention of a disease or disorder which would benefit from an increase in NK cell function or activity. Exemplary diseases and disorders that can be treated using the compositions and methods of the invention include, but are not limited to cancer and infectious diseases.

The following are non-limiting examples of cancers that can be diagnosed or treated by the disclosed methods and compositions: acute lymphoblastic leukemia, acute myeloid leukemia, adrenocortical carcinoma, appendix cancer, basal cell carcinoma, bile duct cancer, bladder cancer, bone cancer, brain and spinal cord tumors, brain stem glioma, brain tumor, breast cancer, bronchial tumors, burkitt lymphoma, carcinoid tumor, central nervous system atypical teratoid/rhabdoid tumor, central nervous system embryonal tumors, central nervous system lymphoma, cerebellar astrocytoma, cerebral astrocytoma/malignant glioma, cerebral astrocytotna/malignant glioma, cervical cancer, childhood visual pathway tumor, chordoma, chronic lymphocytic leukemia, chronic myelogenous leukemia, chronic myeloproliferative disorders, colon cancer, colorectal cancer, craniopharyngioma, cutaneous cancer, cutaneous t-cell lymphoma, endometrial cancer, ependymoblastoma, ependymoma, esophageal cancer, ewing family of tumors, extracranial cancer, extragonadal germ cell tumor, extrahepatic bile duct cancer, extrahepatic cancer, eye cancer, fungoides, gallbladder cancer, gastric (stomach) cancer, gastrointestinal cancer, gastrointestinal carcinoid tumor, gastrointestinal stromal tumor (gist), germ cell tumor, gestational cancer, gestational trophoblastic tumor, glioblastoma, glioma, hairy cell leukemia, head and neck cancer, hepatocellular (liver) cancer, histiocytosis, hodgkin lymphoma, hypopharyngeal cancer, hypothalamic and visual pathway glioma, hypothalamic tumor, intraocular (eye) cancer, intraocular melanoma, islet cell tumors, kaposi sarcoma, kidney (renal cell) cancer, langerhans cell cancer, langerhans cell histiocytosis, laryngeal cancer, leukemia, lip and oral cavity cancer, liver cancer, lung cancer, lymphoma, macroglobulinemia, malignant fibrous histiocvtoma of bone and osteosarcoma, medulloblastoma, medulloepithelioma, melanoma, merkel cell carcinoma, mesothelioma, metastatic squamous neck cancer with occult primary, mouth cancer, multiple endocrine neoplasia syndrome, multiple myeloma, mycosis, myelodysplastic syndromes, myelodysplastic/myeloproliferative diseases, myelogenous leukemia, myeloid leukemia, myeloma, myeloproliferative disorders, nasal cavity and paranasal sinus cancer, nasopharyngeal cancer, neuroblastoma, non-hodgkin lymphoma, non-small cell lung cancer, oral cancer, oral cavity cancer, oropharyngeal cancer, osteosarcoma and malignant fibrous histiocytoma, osteosarcoma and malignant fibrous histiocytoma of bone, ovarian, ovarian cancer, ovarian epithelial cancer, ovarian germ cell tumor, ovarian low malignant potential tumor, pancreatic cancer, papillomatosis, paraganglioma, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm, plasma cell neoplasm/multiple myeloma, pleuropulmonary blastoma, primary central nervous system cancer, primary central nervous system lymphoma, prostate cancer, rectal cancer, renal cell (kidney) cancer, renal pelvis and ureter cancer, respiratory tract carcinoma involving the nut gene on chromosome 15, retinoblastoma, rhabdomyosarcoma, salivary gland cancer, sarcoma, sezary syndrome, skin cancer (melanoma), skin cancer (nonmelanoma), skin carcinoma, small cell lung cancer, small intestine cancer, soft tissue cancer, soft tissue sarcoma, squamous cell carcinoma, squamous neck cancer, stomach (gastric) cancer, supratentorial primitive neuroectodermal tumors, supratentorial primitive neuroectodermal tumors and pineoblastoma, T-cell lymphoma, testicular cancer, throat cancer, thymoma and thymic carcinoma, thyroid cancer, transitional cell cancer, transitional cell cancer of the renal pelvis and ureter, trophoblastic tumor, urethral cancer, uterine cancer, uterine sarcoma, vaginal cancer, visual pathway and hypothalamic glioma, vulvar cancer, waldenstrom macroglobulinemia, and wilms tumor.

### Bacterial Infections

In one embodiment, the infectious disease or disorder is associated with a bacterium. In some embodiments, the bacterium can be from any one of the following phyla: Acidobacteria, Actinobacteria, Aquificae, Bacteroidetes, Caldiserica, Chlamydiae, Chlorobi, Chloroflexi, Chrysiogenetes, Cyanobacteria, Deferribacteres, Deinococcus-Thermus, Dictyoglomi, Elusimicrobia, Fibrobacteres, Firmicutes, Fusobacteria, Gemmatimonadetes, Lentisphaerae, Nitrospira, Planctomycetes, Proteobacteria, Spirochaetes, Synergistetes, Tenericutes, Thermodesulfobacteria, Thermotogae, and Verrucomicrobia.

The bacterium can be a gram-positive bacterium or a gram-negative bacterium. The bacterium can be an aerobic bacterium or an anerobic bacterium. The bacterium can be an autotrophic bacterium or a heterotrophic bacterium. The bacterium can be a mesophile, a neutrophile, an extremophile, an acidophile, an alkaliphile, a thermophile, a psychrophile, an halophile, or an osmophile.

The bacterium can be an anthrax bacterium, an antibiotic resistant bacterium, a disease-causing bacterium, a food poisoning bacterium, an infectious bacterium, Salmonella bacterium, Staphylococcus bacterium, Streptococcus bacterium, or tetanus bacterium. The bacterium can be a mycobacteria, Clostridium tetani, Yersinia pestis, Bacillus anthracis, methicillin-resistant Staphylococcus aureus (MRSA), or Clostridium difficile.

### Viral Infections

In one embodiment, the infectious disease or disorder is associated with a bacterium. In some embodiments, the virus is from one of the following families: Adenoviridae, Arenaviridae, Bunyaviridae, Caliciviridae, Coronaviridae, Filoviridae, Hepadnaviridae, Herpesviridae, Orthomyxoviridae, Papovaviridae, Paramyxoviridae, Parvoviridae, Picornaviridae, Poxviridae, Reoviridae, Retroviridae, Rhabdoviridae, or Togaviridae. The viral antigen can be from human immunodeficiency virus (HIV), Chikungunya virus (CHIKV), dengue fever virus, papilloma viruses, for example, human papillomoa virus (HPV), polio virus, hepatitis viruses, for example, hepatitis A virus (HAV), hepatitis B virus (HBV), hepatitis C virus (HCV), hepatitis D virus (HDV), and hepatitis E virus (HEV), smallpox virus (Variola major and minor), vaccinia virus, influenza virus, rhinoviruses, equine encephalitis viruses, rubella virus, yellow fever virus, Norwalk virus, hepatitis A virus, human T-cell leukemia virus (HTLV-I), hairy cell leukemia virus (HTLV-II), California encephalitis virus, Hanta virus (hemorrhagic fever), rabies virus, Ebola fever virus, Marburg virus, measles virus, mumps virus, respiratory syncytial virus (RSV), herpes simplex 1 (oral herpes), herpes simplex 2 (genital herpes), herpes zoster (varicella-zoster, a.k.a., chickenpox), cytomegalovirus (CMV), for example human CMV, Epstein-Barr virus (EBV), flavivirus, foot and mouth disease virus, lassa virus, arenavirus, severe acute respiratory syndrome-related coronavirus (SARS), Middle East respiratory syndrome-related coronavirus (MERS), severe acute respiratory syndrome-related coronavirus 2 (SARS COV 2) or a cancer causing virus.

### Parasitic Infections

In one embodiment, the infectious disease or disorder is associated with a parasite. In some embodiments, the parasite can be a protozoa, helminth, or ectoparasite. The helminth (i.e., worm) can be a flatworm (e.g., flukes and tapeworms), a thorny-headed worm, or a round worm (e.g., pinworms). The ectoparasite can be lice, fleas, ticks, and mites.

The parasite can be any parasite causing any one of the following diseases: Acanthamoeba keratitis, Amoebiasis, Ascariasis, Babesiosis, Balantidiasis, Baylisascariasis, Chagas disease, Clonorchiasis, Cochliomyia, Cryptosporidiosis, Diphyllobothriasis, Dracunculiasis, Echinococcosis, Elephantiasis, Enterobiasis, Fascioliasis, Fasciolopsiasis, Filariasis, Giardiasis, Gnathostomiasis, Hymenolepiasis, Isosporiasis, Katayama fever, Leishmaniasis, Lyme disease, Malaria, Metagonimiasis, Myiasis, Onchocerciasis, Pediculosis, Scabies, Schistosomiasis, Sleeping sickness, Strongyloidiasis, Taeniasis, Toxocariasis, Toxoplasmosis, Trichinosis, and Trichuriasis.

The parasite can be Acanthamoeba, Anisakis, Ascaris lumbricoides, Botfly, Balantidium coli, Bedbug, Cestoda (tapeworm), Chiggers, Cochliomyia hominivorax, Entamoeba histolytica, Fasciola hepatica, Giardia lamblia, Hookworm, Leishmania, Linguatula serrata, Liver fluke, Loa loa, Paragonimus-lung fluke, Pinworm, Plasmodium falciparum, Schistosoma, Strongyloides stercoralis, Mite, Tapeworm, Toxoplasma gondii, Trypanosoma, Whipworm, or Wuchereria bancrofti.

### Fungal Infection

In one embodiment, the infectious disease or disorder is associated with a fungus. In some embodiments, the fungus can be Aspergillus species, Blastomyces dermatitidis, Candida yeasts (e.g., Candida albicans), Coccidioides, Cryptococcus neoformans, Cryptococcus gattii, dermatophyte, Fusarium species, Histoplasma capsulatum, Mucoromycotina, Pneumocystis jirovecii, Sporothrix schenckii, Exserohilum, or Cladosporium.

In one aspect, the invention provides a method for preventing in a subject, a disease or disorder, by administering to the subject a composition described herein. Administration of a prophylactic agent can occur prior to the manifestation of symptoms characteristic of the disease or disorder, such that the disease or disorder is prevented or delayed in its progression.

In some embodiments, the method comprises administering an effective amount of a composition described herein to a subject diagnosed with, suspected of having, or at risk for developing cancer or an infectious disease or disorder. In one embodiment, the composition is administered systemically to the subject.

The composition of the invention may be administered to a patient or subject in need in a wide variety of ways. Modes of administration include intraoperatively intravenous, intravascular, intramuscular, subcutaneous, intracerebral, intraperitoneal, soft tissue injection, surgical placement, arthroscopic placement, and percutaneous insertion, e.g., direct injection, cannulation or catheterization. Any administration may be a single application of a composition of invention or multiple applications. Administrations may be to single site or to more than one site in the individual to be treated. Multiple administrations may occur essentially at the same time or separated in time.

Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions of the present invention may be administered in a manner appropriate to the disease to be treated (or prevented). The quantity and frequency of administration will be determined by such factors as the condition of the subject, and the type and severity of the subject's disease, although appropriate dosages may be determined by clinical trials.

When “therapeutic amount” is indicated, the precise amount of the compositions of the present invention to be administered can be determined by a physician with consideration of individual differences in age, weight, disease type, extent of disease, and condition of the patient (subject).

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the compositions of the present invention are administered to a patient by intradermal or subcutaneous injection. In another embodiment, the compositions of the present invention are administered by i.v. injection.

The pharmaceutical compositions useful for practicing the invention may be administered to deliver a dose of from 1 ng/kg/day and 100 mg/kg/day. In one embodiment, the invention envisions administration of a dose which results in a concentration of the compound of the present invention from 1 μM and 10 μM in a mammal.

Typically, dosages which may be administered in a method of the invention to a mammal range in amount from 0.5 μg to about 50 mg per kilogram of body weight of the mammal, while the precise dosage administered will vary depending upon any number of factors, including but not limited to, the type of mammal and type of disease state being treated, the age of the mammal and the route of administration. In one embodiment, the dosage will vary from about 1 μg to about 50 mg per kilogram of body weight of the mammal. In one embodiment, the dosage will vary from about 1 mg to about 10 mg per kilogram of body weight of the mammal.

The compound may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

In one embodiment, the invention provides methods of treating or preventing cancer, or of treating and preventing, aiding in the prevention, and/or reducing the risk of an infectious disease in an individual.

One aspect of the invention provides a method of preventing, aiding in the prevention, and/or reducing the risk of HIV infection or a disease or disorder associated therewith (e.g., AIDS).

In some embodiments of treating or preventing an infectious disease in an individual in need thereof, a second agent is administered to the individual, such as an antibody, a CAR molecule, an antigen or an immunogen for promoting an immune response against the infectious disease.

The present invention is further illustrated in the following Examples. It should be understood that these Examples, while indicating exemplary embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Experimental Examples

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out certain embodiments of the present invention, and are not to be construed as limiting in any way the remainder of the disclosure.

### Example 1: Enhancement of IL15/IL15RA Signaling in Immune Cells Using CRISPR-dCas9-VPR Platform

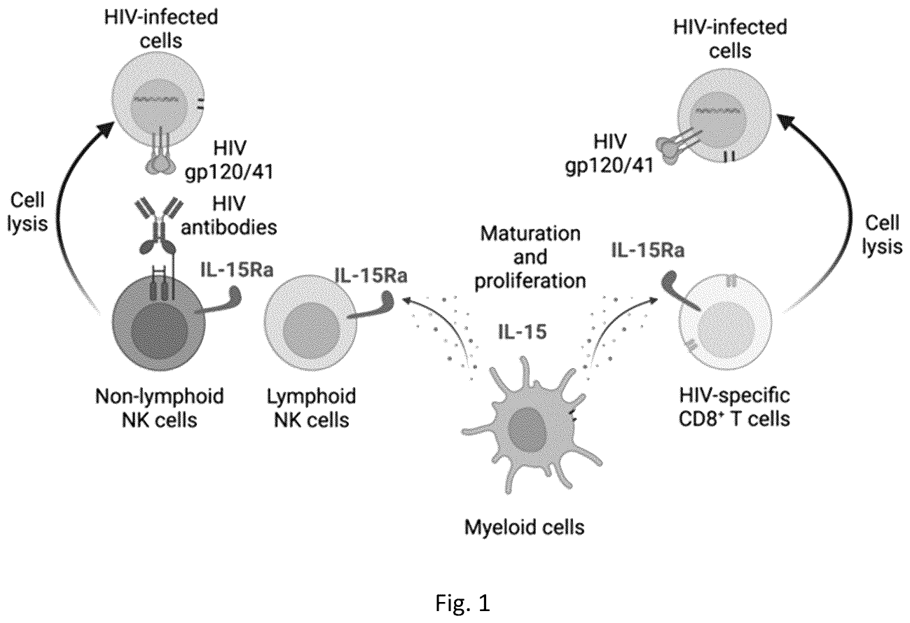

NK (natural killer) and CTLs (cytotoxic T cells) functions are positively regulated by IL-15 signaling. The IL-15 receptor comprises three subunits: IL-15R alpha, beta, and gamma. The IL-15/IL-15RA dimer can be trans-presented by myeloid cells to CD4+ and CD8+ cells and NK cells, which express the beta and gamma subunits. In addition, CD8+ T cells and NK cells can also express IL-15RA upon stimulation and trigger IL-15 signaling through cis-presentation. Specific upregulation of IL-15/IL-15RA expression/signaling in CTLs and NK cells improves their proliferation and cytolytic functions and thus lead to better clearance of HIV-infected cells (FIG. 1). CRISPR-Cas9 platforms using catalytically dead Cas9 (dCas9) protein allow precise control of genome expression without gene editing. Here to accomplish potent transactivation of the transcription of IL-15RA and IL-15 in immune cells, the VPR platform was utilized where dCas9 is fused to linked in tandem VP64-p65-Rts transcription activation domains.

Design and Selection of CRISPR gRNAs

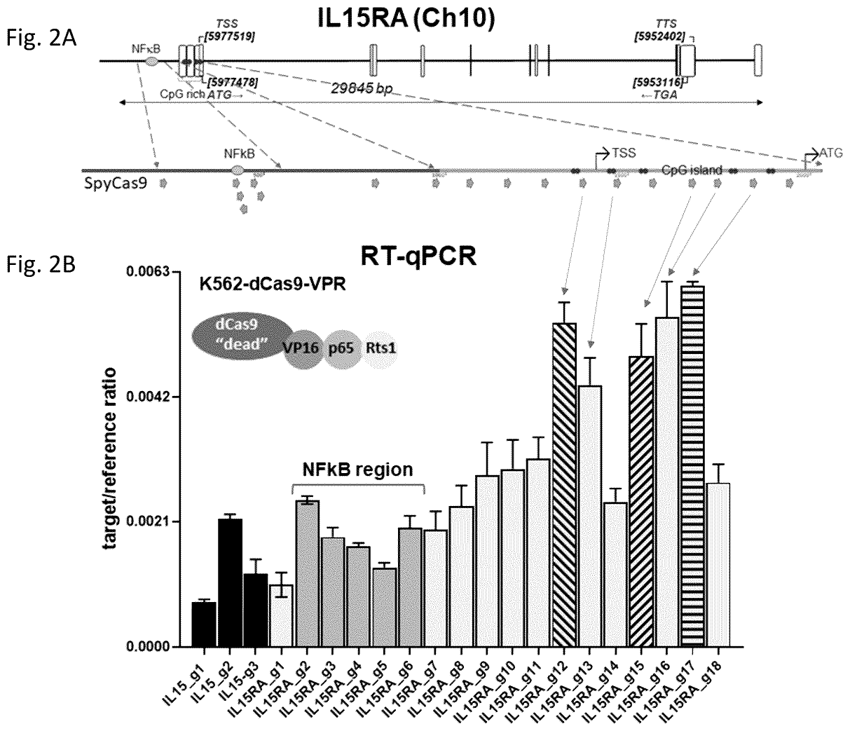

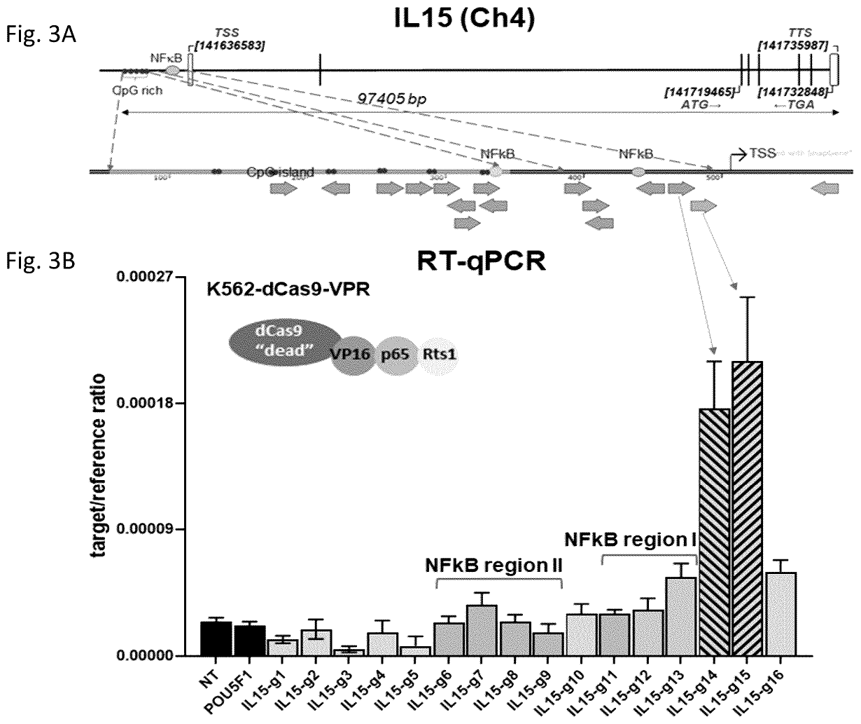

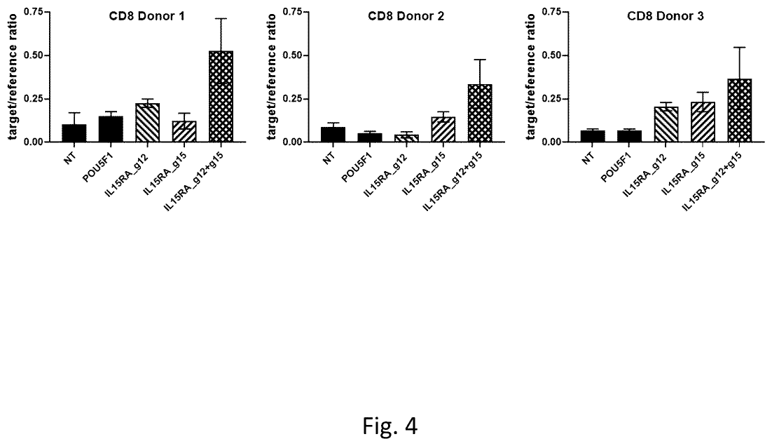

The human IL-15Ra (chromosome 10) and IL-15 (chromosome 4) gene coding sequences including promoter regions upstream of transcription start sites were screened using the CRISPR design tool (CRISPOR) for the presence of gRNAs binding sites for SpCas9 (NGG PAM sequence). A set of 18 candidate gRNAs targeting the promoter of the IL-15Ra gene (FIG. 2A and Table 1) and candidate 16 gRNAs the targeting promoter of the IL-15 gene (FIG. 3A and Table 2) having the highest ON-target efficiency and the lowest OFF-target scores were selected.

TABLE 1
CRISPR gRNAs targeting human IL-15Ra gene promoter region

| SEQ ID | gRNA | | Target site sequence |
|---|---|---|---|
| NO: | # | gRNA name | (21 n + PAM) |
| 1 | g1 | IL15Ra_237forw | TTACAAAGAGTCTGTAATAATGG |
| 2 | g2 | IL15Ra_437forw | TAATGTGGGATTTCCCCAGTTGG |
| 3 | g3 | IL15Ra_448forw | TTCCCCAGTTGGAGTAAGGGTGG |
| 4 | g4 | IL15Ra_432rev | ATAGCCACCCTTACTCCAACTGG |
| 5 | g5 | IL15Ra_486forw | AAAAAAGAATGGACCTTGTGTGG |
| 6 | g6 | IL15Ra_504forw | TGTGGTCACTGCCGGATGGTAGG |
| 7 | g7 | IL15Ra_819forw | TCAGGAGTGGCCACCGCGCTCGG |
| 8 | g8 | IL15Ra_985forw | ACACGTTGTGAGATTTTAAGAGG |
| 9 | g9 | IL15Ra_1078forw | AGCTTACAGACCCGGTTCTCAGG |
| 10 | g10 | IL15Ra_1181forw | GGCTCAGAGGTGAAGCACTGTGG |
| 11 | g11 | IL15Ra_1284forw | ATTCGGCGAAGTGGCGGAGCTGG |
| 12 | g12 | IL15Ra_1388forw | GTGCCCGCGCCTCCGCACCGCGG |
| 13 | g13 | IL15Ra_1482forw | AAAGCCGAAGGCGGAGCAGCTGG |
| 14 | g14 | IL15Ra_1580forw | TTTGCCCACTCTCTTCGCAGTGG |
| 15 | g15 | IL15Ra_1688forw | TCCTGGGTCCCCACCGGGCACGG |
| 16 | g16 | IL15Ra_1758forw | GGCGGGGCTCCTTCCCCAGCAGG |
| 17 | g17 | IL15Ra_1856forw | AGCGAATGCGACTGGCGGGGCGG |
| 18 | g18 | IL15Ra_1955forw | GAGCTGCCGCCATGGCCCCGCGG |

TABLE 2
Candidate CRISPR gRNAs targeting
human IL-15 gene promoter region

| SEQ | | | |
|---|---|---|---|
| ID | gRNA | | Target site sequence |
| NO: | # | gRNA name | (21 n + PAM) |
| 19 | g1 | IL15_194forw | TTTCCTTCTTGCAGCCTGTGCGG |
| 20 | g2 | IL_15_208rev | GCTGGGCGGCTACTCTAGACCGG |
| 21 | g3 | IL15_271forw | TCCCTTTCCTCAGGAAGACTTGG |
| 22 | g4 | IL15_292forw | GGATGCGCGCCAGCCCAGCCCGG |
| 23 | g5 | IL15_299rev | GAAGCTTCCGCAGCTGAACCTGG |
| 24 | g6 | IL15_312forw | CGGGTCTCCAGGTTCAGCTGCGG |
| 25 | g7 | IL15_322rev | CCCAAGCGACGGGCACCATGTGG |
| 26 | g8 | IL15_327forw | AGCTGCGGAAGCTTCCCACATGG |
| 27 | g9 | IL15_341forw | CCCACATGGTGCCCGTCGCTTGG |
| 28 | g10 | IL15_399rev | CCTGCAGGAACACCAGCCACCGG |
| 29 | g11 | IL15_407forw | TAGAGGAGGAGACCGGTGGCTGG |
| 30 | g12 | IL15_420forw | CGGTGGCTGGTGTTCCTGCAGGG |
| 31 | g13 | IL15_436rev | GGGCAAGCGCAAGGACATCGAGG |
| 32 | g14 | IL15_482forw | AAGTTCTGCGCCCCAGGCCCCGG |
| 33 | g15 | IL15_498forw | GCCCCGGCAGGGACGGAAACAGG |
| 34 | g16 | IL15_562rev | AAACACCTCCCGCGGAGACTGGG |

Testing of CRISPR gRNAs Targeting IL15Ra and IL15 in K562-dCas9-VPR Cell Line

K562 lymphoblast cell line stably expressing dCas9-VPR (Horizon) was electroporated with synthetic gRNAs, including non-targeting gRNA as a control. After three days, the mRNA expression of target genes was examined by RT-qPCRs. Experiments were repeated one more time. Based on the results lead gRNAs showing the highest and repeatable induction of the target gene expression were identified: g12, g15, and g17 for IL-15Ra (FIG. 2B, highlighted grey in Table 1) and g14 and g15 for IL-15 promoter (FIG. 3B, highlighted grey in Table 2).

Validation of Lead CRISPR gRNAs in Primary CD8+ T Cells

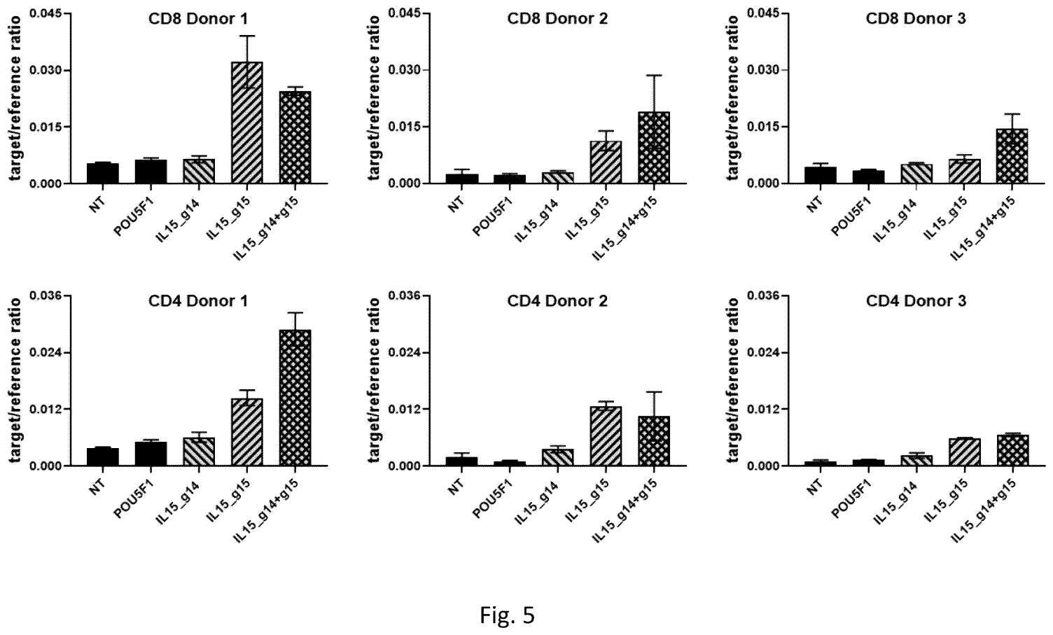

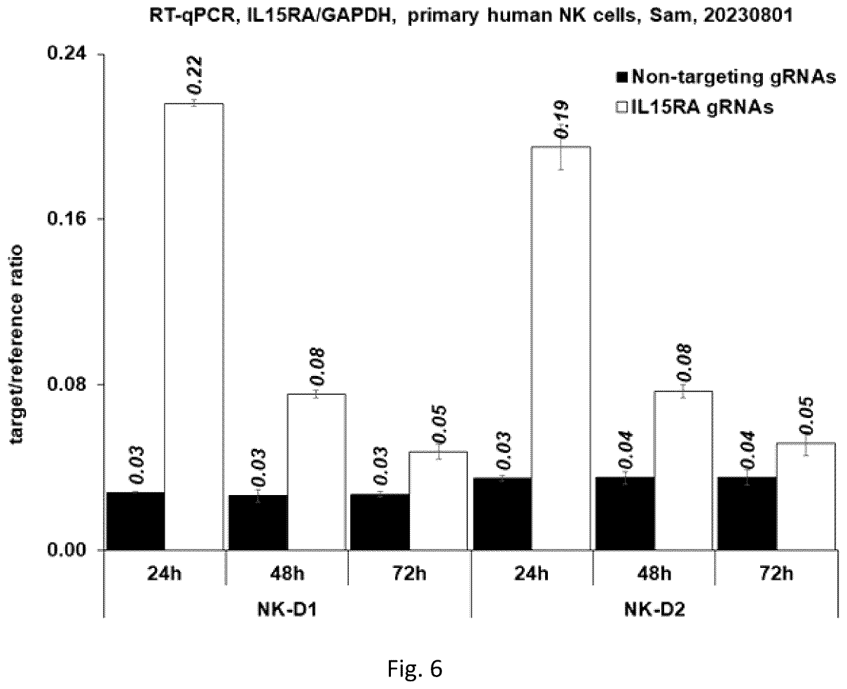

The lead gRNAs were co-transfected with mRNA for dCas9-VPR (Horizon) into primary human CD8+ T-cells isolated from the blood of three healthy donors (de-identified) single or together. As shown in FIG. 4 combining g12+g15 resulted in stronger induction of IL-15Ra transcription than single gRNAs. Similarly, combining g14+g15 resulted in in stronger induction of IL-15 transcription in primary CD8+ T-cells (FIG. 5).

Validation of Lead CRISPR gRNAs in Primary NK Cells

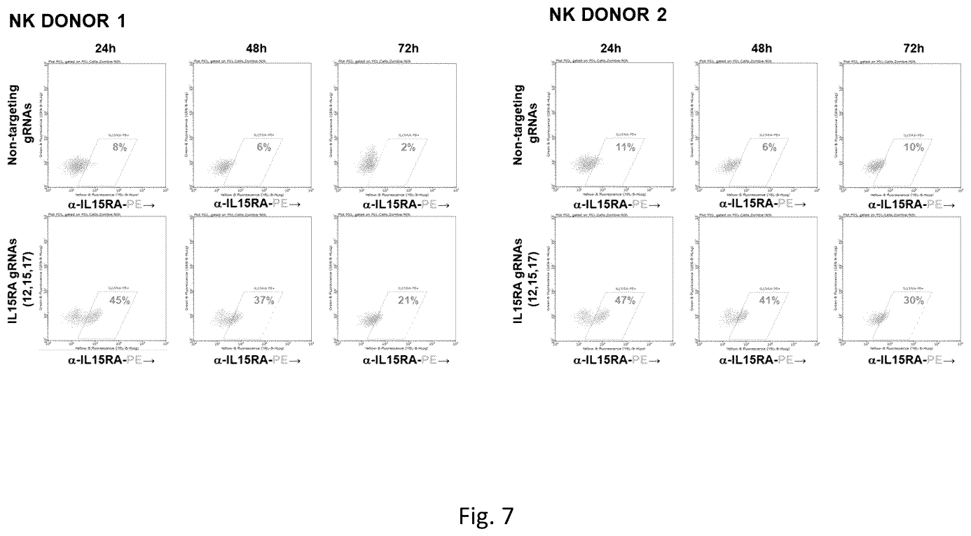

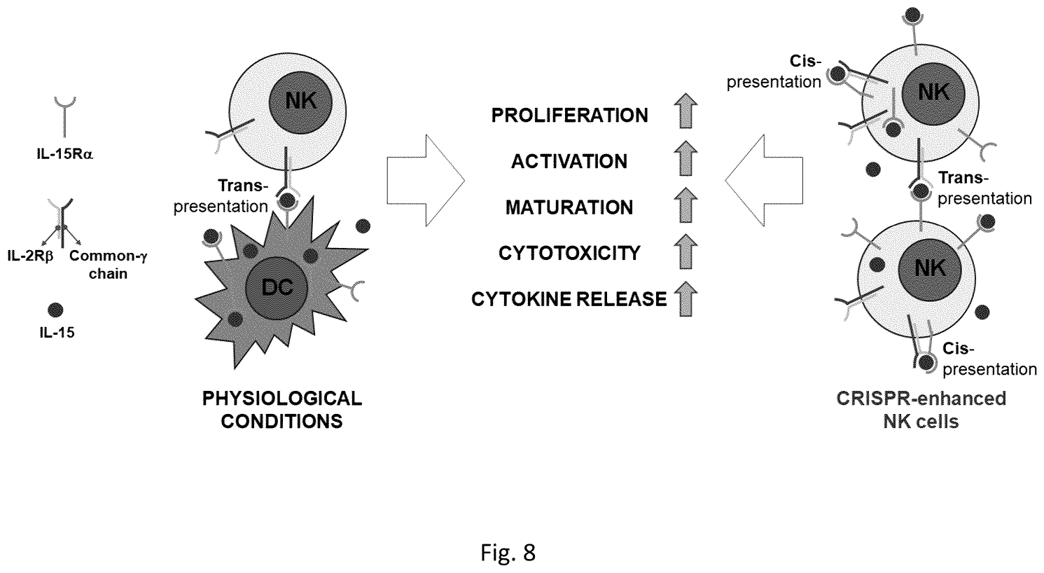

Finally, gRNAs targeting IL-15Ra were examined in primary human NK cells isolated from two healthy donors. Here, a mixture of g12+g15+g17 was used and cells were harvested for RNA and flow cytometry analysis after 24 h, 48 h, and 72 h post transfection. The gRNAs mix was able to induce IL-15Ra mRNA (FIG. 6) and cell surface expression (FIG. 7) at all time points with the strongest effect seen 24 h after transfection. The identified and validated in vitro lead CRISPR gRNAs allowing dCas9-VPR-mediated induction of IL-15Ra and IL-15 genes expression in CTLs and NKs are highlighted/underscored in Table 1 and Table 2.

Soluble monomer IL-15 or heterodimeric complex IL-15/IL-15RA are currently being evaluated in phase I/II clinical trials for cancer. It was shown that prolonged or repeated exposure to IL-15/IL-15RA and high doses lead to detrimental dysfunction of NK cells and CTLs. This approach uses a natural way of inducing IL-15RA and IL-15 expression in immune cells by targeting promoter regions of their respective genes which should result in the transient production of physiological levels of both proteins. Simultaneous induction of both IL-15 and IL-15RA in the target cells allows stimulation of the cells through both cis (self-stimulation) and trans (through adjacent cells) mechanisms. Soluble IL-15/IL-15RA works only through the trans mechanism. By inducing IL-15RA in the target CTL and NK cells we release their dependence on trans-activation by IL-15/IL-15RA-producing myeloid cells. dCas9-VPR/gRNAs delivery can be customized to specifically target CTLs and NK cells reducing the risk of adverse effects related to systemic administration and global activation of the immune system.

Functional restoration of HIV-specific cytotoxic T lymphocytes (CTLs) and natural killer (NK) cells is required for cytotoxic clearance of residual HIV-infected cells. CTLs and NKs mediate strong suppression of HIV infection. Chronic HIV-1 infection drives functional exhaustion of antigen-specific CTLs and NK cells. HIV-specific CTLs and NK cells are functionally impaired with poor cytolytic capacity which cannot be restored by ART. Thus, functional restoration of broad CTL and NK cell response is needed to clear latent HIV infection. IL-15 and IL-15RA/IL-15 conjugate agonists are currently being evaluated for treatment of HIV-1 and cancer. The present invention uses a CRISPR-based approach to specifically upregulate the expression of IL-15Ra and IL-15 in HIV-specific CTLs and NK cells to enhance cytotoxic-cell-mediated reservoir clearance. Additionally, it can be used in combination with latency reversal agents to improve the “kick and kill” strategy. Finally, it can enhance vaccine-elicited immunity by augmenting the number, function, maintenance, and longevity of vaccine-induced cellular immune responses (HIV-1 vaccines, broadly neutralizing anti-HIV-1 antibody-based therapies). The same approach, CRISPR-mediated upregulation of IL-15RA/IL-15 signaling in cytotoxic immune cells can be applied to other than HIV-1 chronic viral infections such as HBV, HCV, HTLV-1, and influenza. Additionally, non-viral chronic infections associated with CTL exhaustion or NK cell dysfunction such as tuberculosis, Listeria, malaria, toxoplasmosis, and leishmaniasis. Also, as mentioned above it can enhance vaccine-elicited immunity by augmenting the number, function, maintenance, and longevity of vaccine-induced cellular immune responses. This approach can be used to improve the functions of CAR-T-cells and cytotoxic clearance of cancer cells. As mentioned above, it can enhance vaccine-elicited immunity (cancer vaccines).

### Example 2: ACTIVATION OF NK CELLS USING IL-15/IL-15Ra TARGETED dCAS9-VPR

Immune dysregulation and dampened activity of immune cells is a hallmark of chronic HIV infection. NK cells largely rely on IL-15 signaling for the development and maintenance of populations in a physiological setting, and dysregulated IL-15 signaling in NK cells is observed in people living with HIV. Physiologically, IL-15 and IL-15Ra dimerize intracellularly and are trans-presented by myeloid cells to NK cells, which express the beta and gamma subunits. NK cells do not normally express IL-15 cytokine nor IL-15Ra but have been shown to be responsive to IL-15 signaling through cis-presentation if made to express IL-15 and IL-15Ra themselves in vitro. Increasing IL-15/IL-15Ra expression/signaling in NK cells should improve their proliferative and cytolytic abilities and lead to better clearance of HIV-infected cells. CRISPR-Cas9 platforms using catalytically dead Cas9 (dCas9) protein allow precise control of gene expression without genome editing. To increase IL-15Ra/IL-15 expression in NK cells, we utilized the dCas9-VPR platform, where dCas9 is linked to a tripartite VP64-p65-Rts transcription activating domain.

### The Methods are Now Described

Promoter region sequences of IL-15 and IL-15Ra were analyzed for gRNA binding sites, and gRNAs were identified for evaluation. K562 lymphoblast cell line stably expressing dCas9-VPR was electroporated with synthetic gRNAs, including non-targeting gRNA as a control. After three days, the mRNA expression of target genes was examined by qRT-PCR. gRNAs with the highest transcription induction were selected to be further used in combination. This combination of gRNAs in addition to dCas9-VPR mRNA was used to electroporate primary NK cells isolated from healthy blood donors. These cells were then analyzed for IL-15/IL-15Ra expression as well as for presence of known NK cell activation markers via immunolabeling. Additional screening involving metabolic analysis as well as functional assessment of proliferative ability and cytotoxicity will be performed in the near future.

### The Experimental Results are Now Described

Regions in the promoters of IL-15Ra and IL-15 were identified that best responded to dCas9-VPR-mediated transcription activation. gRNAs recruiting dCas9-VPR closest to transcription start sites showed the most potent induction of the target genes transcription. The process was shown to work similarly in primary cells as it did in the K562 dCas9-VPR cell line, with a marked increase in surface expression of IL-15Ra being identified. The functional phenotype of treated cells was classified in the context of their ability to proliferate as well as perform cytotoxic attack.

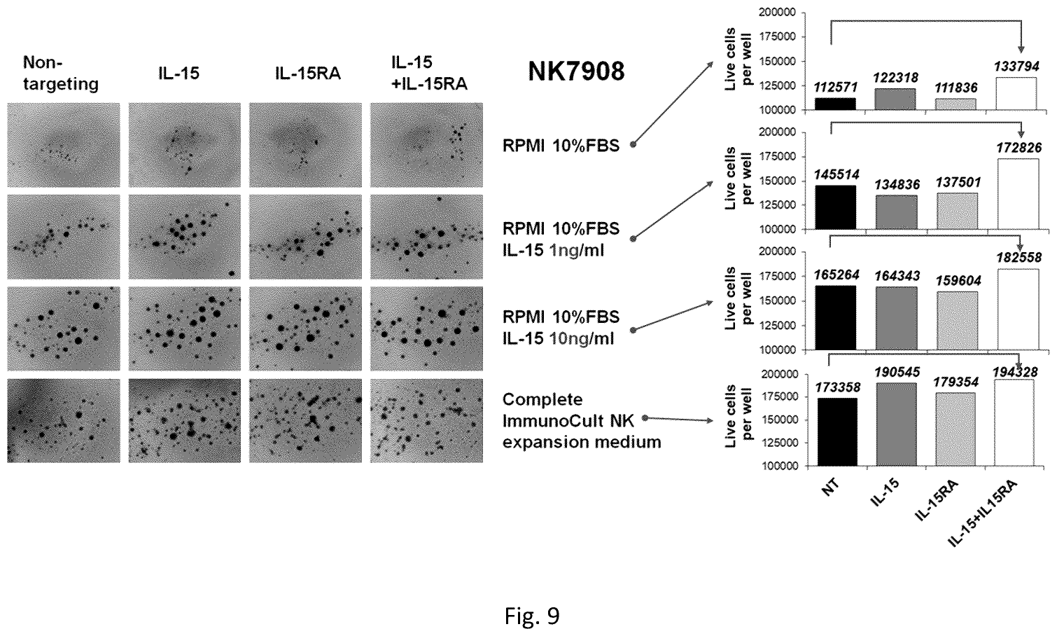

It was hypothesized that upregulation of IL-15Ra in NK cells can enhance IL-15 cis- and trans-presentation and improve their persistence and effector functions when IL-15 is provided or overexpressed. An outline of the hypothesis is provided in FIG. 8.

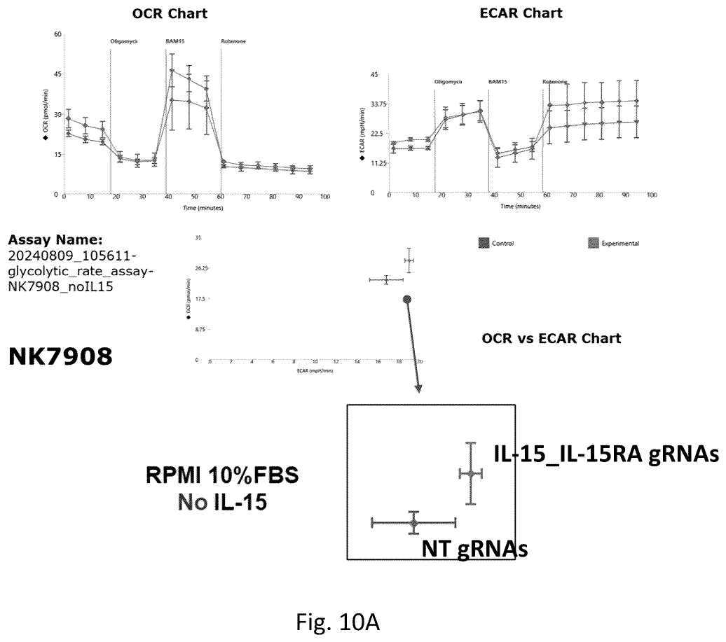

FIG. 9 depicts representative data demonstrating concomitant dCas9-VPR/gRNA-mediated induction of IL-15 and IL-15Ra expression leads to increased proliferation and survival of primary human NK cells in vitro. Primary human NK cells were isolated from the blood of healthy donor using EasySep CD56 positive selection kit (STEMCELL Technologies) and expanded for two weeks in ImmunoCult medium. Next, cells were electroporated with mRNA for dCas9-VPR (Horizon Biosciences) and non-targeting gRNAs (NT), IL-15 promoter targeting gRNAs, IL-15Ra promoter targeting gRNAs or the mix of both IL-15+IL-15Ra promoters targeting gRNAs. 24 h later cells were counted and plated at the density of 10⁶ cells/ml in RPMI medium supplemented with 10% FBS without or with 1 ng/ml or 10 ng/ml of recombinant IL-15 or in complete ImmunoCult medium. After three days the microscopic pictures of the culture wells were taken (left) and cells were stained with viability dye Propidium Iodide and live cells (PI-negative) were counted on Guava MiniCyte (right). The number of live cells was highest in the wells containing cells treated with the mix of both IL-15+IL-15Ra promoters targeting gRNAs (white bars) compared cells treated with non-targeting gRNAs (NT, black bars) or treated with gRNAs targeting IL-15 promoter only (dark grey bars) or IL-15Ra promoter only (light grey bars). Although this increase of viable cell numbers was observed in all culture conditions, the most robust effects were observed in the absence or suboptimal concentration (1 ng/ml) of IL-15 in the growth medium.

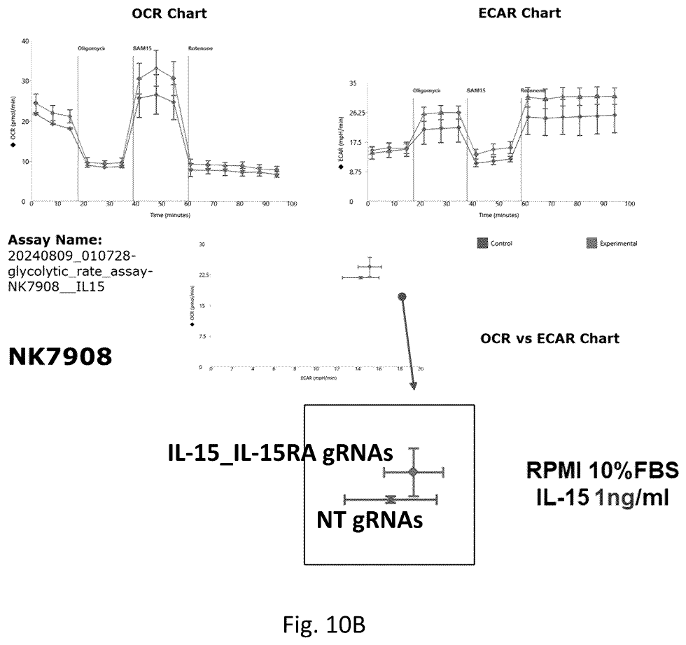

FIG. 10A and FIG. 10B depict concomitant dCas9-VPR/gRNA-mediated induction of IL-15 and IL-15Ra expression leads to increased metabolic activity of primary human NK cells in vitro. Primary human NK cells treated with dCas9-VPR and non-targeting (blue-Control) or the mix of IL-15+IL-15Ra promoters targeting gRNAs (green-Experimental) and cultured for three days without (FIG. 10A) or with suboptimal concentration of IL-15 (1 ng/ml, FIG. 10B) were used NK cell metabolic profiling assay (Agilent Seahorse XFp). Cells treated with the mix of both IL-15+IL-15Ra promoters targeting gRNAs (green-Experimental) show higher metabolic activity (glycolytic and mitochondrial activity) compared to cells treated with non-targeting gRNAs (blue-Control). The effects were more pronounced in the absence of IL-15 in the growth medium (FIG. 10A). OCR stands for oxygen consumption rate, which is proportional to mitochondrial respiration. ECAR stands for extracellular acidification rate and measures mitochondrial oxidative phosphorylation based on the OCR, through real-time and live cell analysis.

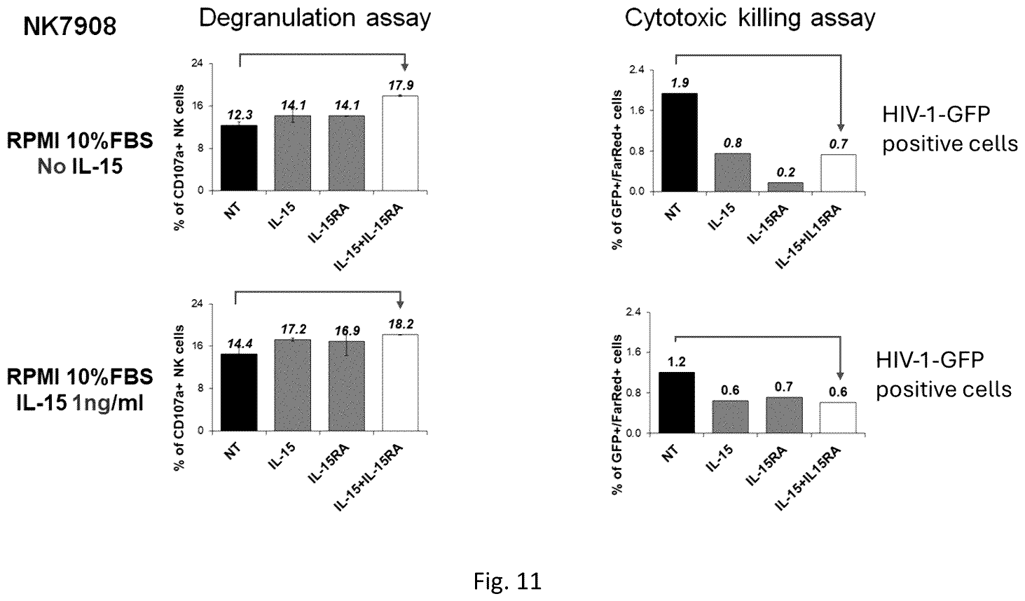

FIG. 11 depicts representative data demonstrating concomitant dCas9-VPR/gRNA-mediated induction of IL-15 and IL-15Ra expression leads to increased cytolytic activity of primary human NK cells in vitro. Primary human NK cells treated with non-targeting gRNAs (NT), IL-15 promoter targeting gRNAs, IL-15Ra promoter targeting gRNAs or the mix of both IL-15+IL-15Ra promoters targeting gRNAs and cultured for three days without (top) or with a suboptimal concentration of IL-15 (1 ng/ml, bottom) were used in degranulation (left) and cytotoxic killing assays (right). For the degranulation assay NK cells were incubated with anti-CD107a-FITC antibody (a marker of degranulation=cytolytic granule release) and FarRed stained K562 target cells in the ratio NK:K562 5:1 for 1 h and then incubated with Monensin (to prevent quenched fluorescence) for 4 h and analyzed for CD107a-FITC positivity using Guava MiniCyte. As shown in A, the number of CD107a positive NK cells (=releasing cytolytic granules) was the highest in the wells containing cells treated with the mix of both IL-15+IL-15Ra promoters targeting gRNAs (white bars) compared cells treated with non-targeting gRNAs (NT, black bars) or treated with gRNAs targeting IL-15 promoter only or IL-15Ra promoter only (grey bars). In parallel, the same NK cells were incubated with isogenic HIV-1-GFP infected CD4+ T target cells in the ratio NK:CD4+ T cells 5:1. After four hours cells were collected, fixed with 2% paraformaldehyde and analyzed for GFP positivity using Guava MiniCyte. As shown in B, the greater decrease of GFP positive (=HIV-1-infected) CD4+ T cells was observed in wells containing NK cells treated with IL-15, IL-15Ra (grey bars) and both IL-15+IL-15Ra (white bars) promoters targeting gRNAs compared to wells with NK cells treated with non-targeting gRNAs (black bars).

### Example 3: BOOSTING NK CELL EFFECTOR FUNCTIONALITY VIA IL-15/IL-15Ra-PROMOTER TARGETED dCAS9-VPR

NK cells, like many other immune cell types, show lessened effector activity during chronic HIV infection. NK cells rely on IL-15 signaling for the development and maintenance of populations physiologically, and dysregulated IL-15 signaling is observed in people living with HIV. IL-15 and IL-15Ra dimerize intracellularly and are trans-presented by myeloid cells to NK cells, which express beta and gamma subunits. NK cells do not express IL-15 cytokine nor IL-15Ra but have been shown to be responsive to IL-15 signaling through cis-presentation if made to express IL-15 and IL-15Ra in vitro. Increasing IL-15/IL-15Ra expression/signaling in NK cells should improve their proliferative and effector abilities leading to better clearance of HIV reservoir. The dCas9-VPR platform uses catalytically dead Cas9 to allow precise control of gene expression without genome editing and was therefore used to increase IL-15Ra/IL-15 expression. gRNA binding sites in the promoter regions of IL-15 and IL-15Ra that best induced transcription in both a dCas9-VPR K562 cell line as well as primary NK cells were identified. gRNAs recruiting dCas9-VPR closest to the transcription start sites most potently induced transcription of the target genes. Treated primary NK cells showed significantly increased transcription levels of both IL-15 and IL-15Ra via RT-qPCR, a marked increase in surface expression of IL-15Ra via flow cytometry was also identified. We have also begun to classify the functional phenotype of treated cells in the context of their ability to proliferate, metabolic state, as well as perform cytotoxic attack. Lastly, as the described dCas9-VPR/gRNA delivery method is based upon a single dosage of RNA the effect is transient. Hence, we are currently investigating methods to achieve stable expression of dCas9-VPR and accompanying gRNA to allow for a more robust and prolonged upregulation and subsequent NK cell activation.

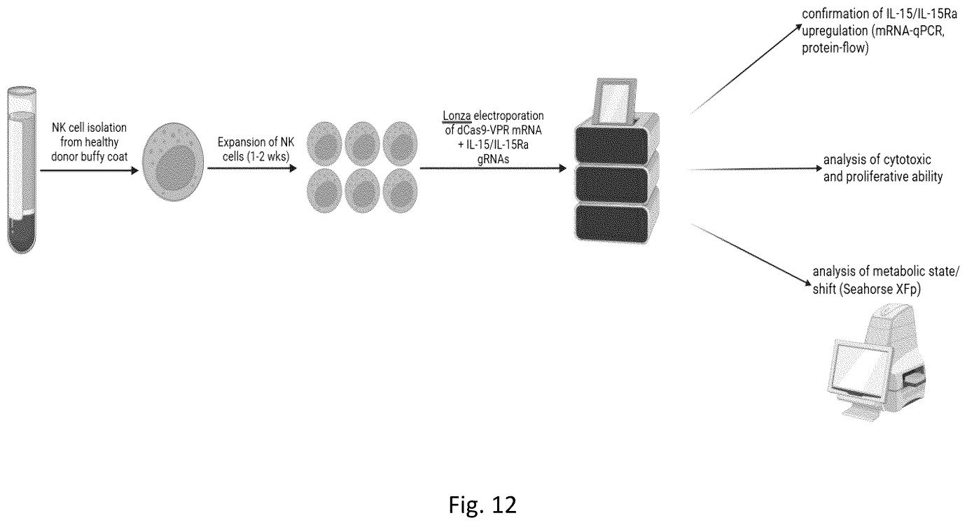

FIG. 12 shows an example of the Primary NK Cell Experimental Workflow.

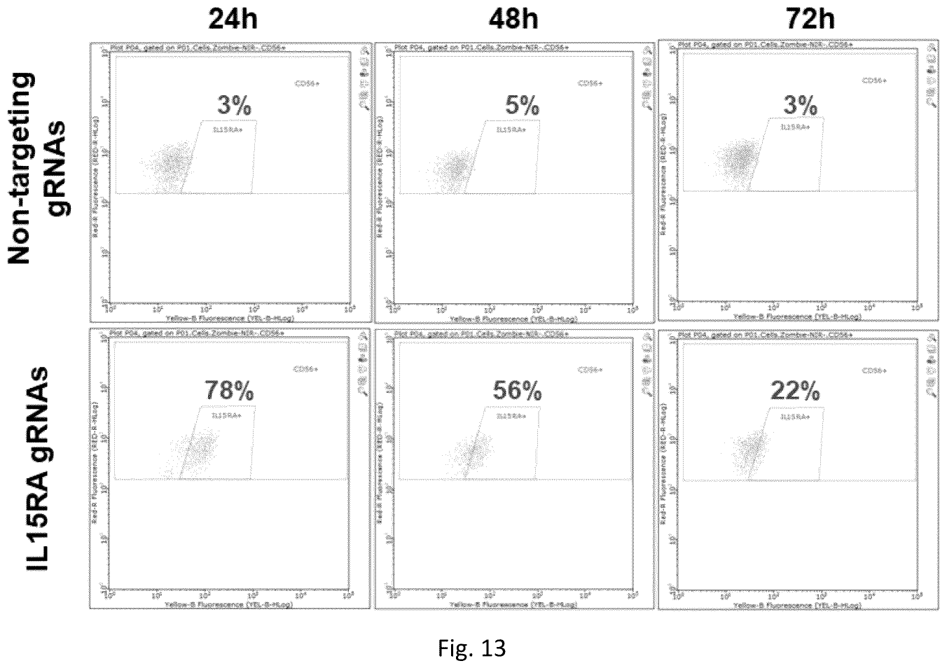

FIG. 13 depicts the validation of lead CRISPR gRNAs targeting IL-15Ra gene promoter in primary human NK cells. Results of immunolabeling/flow cytometry and RT-qPCR. Primary human NK cells were isolated from the blood of two different healthy donors and electroporated with dCas9-VPR mRNA together with lead CRISPR gRNAs targeting human IL-15Ra gene promoter. Every day for three days cells were immunolabeled with anti-IL-15Ra-PE antibody and analyzed for surface expression of IL-15Ra as well as had RNA extracted for RT-qPCR.

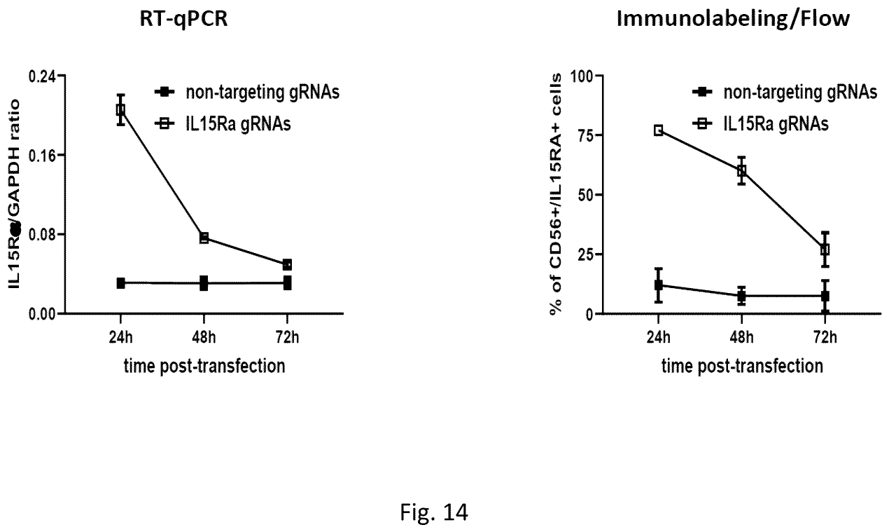

FIG. 14 depicts a summary of the data in FIG. 13.

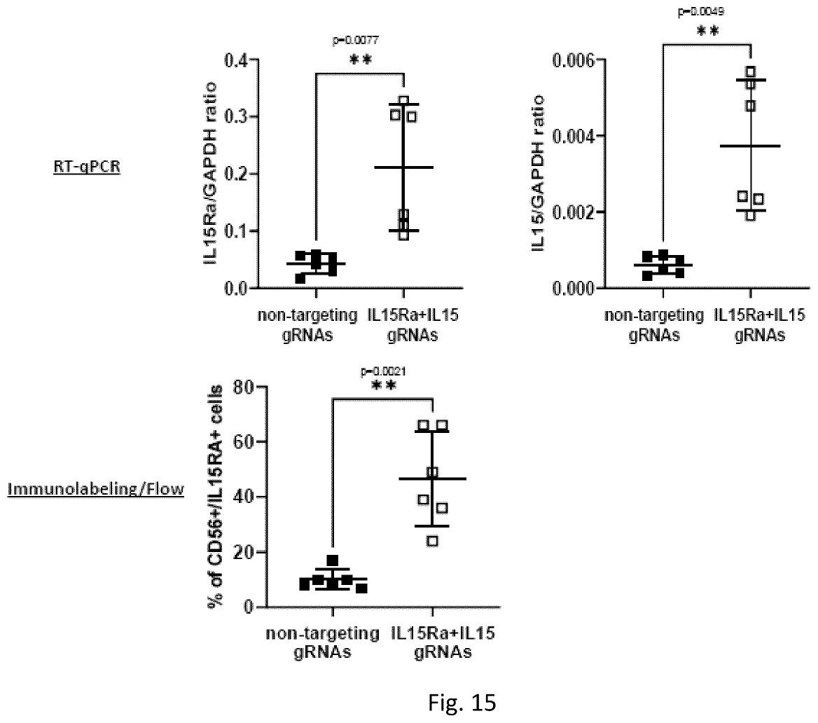

FIG. 15 demonstrates the induction of IL-15/IL-15Ra expression in primary human NK cells. Immunolabeling/flow cytometry (IL-15Ra only) (bottom left) and RTqPCR data (top) collected from NK cells isolated from 6 different blood donors. All data points collected 24 h after electroporation. RNA levels (evaluated with TaqMan probes) for both IL-15 and IL-15Ra were significantly elevated in cells receiving IL-15+IL-15Ra targeting gRNAs, showing proof of concept for the multiplex induction of the two genes from the same treatment in primary NK cells.

Regions in the promoters of IL-15 and IL-15Ra that best responded to dCas9-VPR-mediated transcription activation were identified. gRNAs recruiting dCas9-VPR closest to transcription start sites showed the most potent induction of the target genes transcription. The process was shown to work similarly in primary NK cells as it did in the K562 dCas9-VPR cell line, with a marked increase in surface expression of IL-15Ra being identified.

### Example 4: Macaque NK Cells

A CD3-depleted lymph node explant culture was used for expansion of Macaque NK cells (#KE26) 24 h. Human equivalent gRNAs targeting IL-15 and IL-15Ra promoters are functional in primary macaque NK cells.

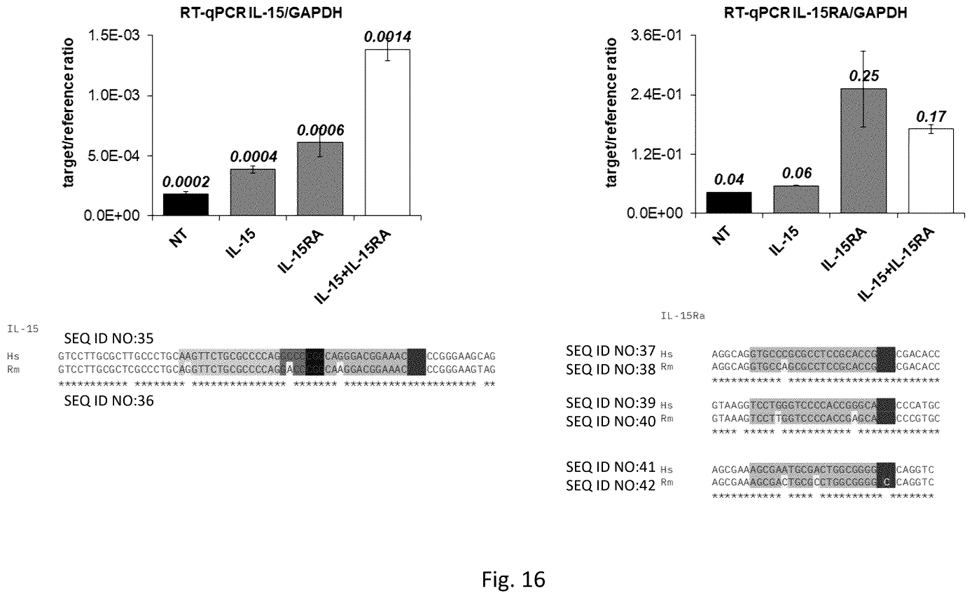

FIG. 16 shows the redesigned macaque specific gRNAs for studies using macaque NK cells.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A genome editing system comprising a targeted transcriptional activator comprising a catalytically dead CRISPR Cas protein linked to a transcription activation domain, and at least one selected from:
a) at least one sgRNA molecule comprising nucleotide sequence that is complementary to the promoter of IL-15Ra, and
b) at least one sgRNA molecule comprising nucleotide sequence that is complementary to the promoter of IL-15.

2. The genome editing system of claim 1, wherein the at least one sgRNA molecule comprising nucleotide sequence that is complementary to the promoter of IL-15Ra is selected from the group consisting of SEQ ID NO: 1-18.

3. The genome editing system of claim 1, wherein the at least one sgRNA molecule comprising nucleotide sequence that is complementary to the promoter of IL-15 is selected from the group consisting of SEQ ID NO: 19-34.

4. The genome editing system of any one of claims 1-3, wherein the system comprises an mRNA molecule encoding the catalytically dead CRISPR Cas protein linked to a transcription activation domain.

5. The genome editing system of any one of claims 1-4, wherein the transcription activation domain comprises a fusion protein comprising VP16, p65 and Rta.

6. The genome editing system of any one of claims 1-5, comprising a combination of:
a) at least one sgRNA molecule comprising nucleotide sequence that is complementary to the promoter of IL-15Ra, and
b) at least one sgRNA molecule comprising nucleotide sequence that is complementary to the promoter of IL-15.

7. A method of increasing the expression of the combination of IL-15Ra and IL-15 in a subject, comprising administering to the subject:
a) a genome editing system of any one of claims 1-6 comprising a targeted transcriptional activator comprising a catalytically dead CRISPR Cas protein linked to a transcription activation domain,
b) at least one sgRNA molecule comprising nucleotide sequence that is complementary to the promoter of IL-15Ra, and
c) at least one sgRNA molecule comprising nucleotide sequence that is complementary to the promoter of IL-15.

8. The method of claim 7, wherein the method comprises administering a first composition comprising:
a) a genome editing system of any one of claims 1-6 comprising a targeted transcriptional activator comprising a catalytically dead CRISPR Cas protein linked to a transcription activation domain, and
b) at least one sgRNA molecule comprising nucleotide sequence that is complementary to the promoter of IL-15Ra, and
a second composition comprising:
c) a genome editing system of any one of claims 1-6 comprising a targeted transcriptional activator comprising a catalytically dead CRISPR Cas protein linked to a transcription activation domain, and
d) at least one sgRNA molecule comprising nucleotide sequence that is complementary to the promoter of IL-15.

9. A method of increasing the proliferation of immune cells in a subject, comprising administering to the subject:
a) a genome editing system of any one of claims 1-6 comprising a targeted transcriptional activator comprising a catalytically dead CRISPR Cas protein linked to a transcription activation domain,
b) at least one sgRNA molecule comprising nucleotide sequence that is complementary to the promoter of IL-15Ra, and
c) at least one sgRNA molecule comprising nucleotide sequence that is complementary to the promoter of IL-15.

10. The method of claim 9, wherein the method comprises administering a first composition comprising:
a) a genome editing system of any one of claims 1-6 comprising a targeted transcriptional activator comprising a catalytically dead CRISPR Cas protein linked to a transcription activation domain, and
b) at least one sgRNA molecule comprising nucleotide sequence that is complementary to the promoter of IL-15Ra, and
a second composition comprising:
c) a genome editing system of any one of claims 1-6 comprising a targeted transcriptional activator comprising a catalytically dead CRISPR Cas protein linked to a transcription activation domain, and
d) at least one sgRNA molecule comprising nucleotide sequence that is complementary to the promoter of IL-15.

12. The method of claim 10, wherein the immune cell is selected from the group consisting of a T cell, natural killer (NK) cell, myeloid cell, antigen presenting cell, dendritic cell, macrophage, and B cell.

13. The method of claim 12, wherein the immune cell is a cytotoxic T lymphocyte or an NK cell.

14. A method of increasing the cytotoxicity of immune cells in a subject, comprising administering to the subject:
a) a genome editing system of any one of claims 1-6 comprising a targeted transcriptional activator comprising a catalytically dead CRISPR Cas protein linked to a transcription activation domain,
b) at least one sgRNA molecule comprising nucleotide sequence that is complementary to the promoter of IL-15Ra, and
c) at least one sgRNA molecule comprising nucleotide sequence that is complementary to the promoter of IL-15.

15. The method of claim 14, wherein the method comprises administering a first composition comprising:
a) a genome editing system of any one of claims 1-6 comprising a targeted transcriptional activator comprising a catalytically dead CRISPR Cas protein linked to a transcription activation domain, and
b) at least one sgRNA molecule comprising nucleotide sequence that is complementary to the promoter of IL-15Ra, and
a second composition comprising:
c) a genome editing system of any one of claims 1-6 comprising a targeted transcriptional activator comprising a catalytically dead CRISPR Cas protein linked to a transcription activation domain, and
d) at least one sgRNA molecule comprising nucleotide sequence that is complementary to the promoter of IL-15.

16. The method of claim 14, wherein the immune cell is a cytotoxic T lymphocyte or a natural killer (NK) cell.

17. A method of treating or preventing a disease or disorder in a subject, comprising administering to the subject:
a) a genome editing system of any one of claims 1-6 comprising a targeted transcriptional activator comprising a catalytically dead CRISPR Cas protein linked to a transcription activation domain,
b) at least one sgRNA molecule comprising nucleotide sequence that is complementary to the promoter of IL-15Ra, and
c) at least one sgRNA molecule comprising nucleotide sequence that is complementary to the promoter of IL-15.

18. The method of claim 17, wherein the method comprises administering a first composition comprising:
a) a genome editing system of any one of claims 1-6 comprising a targeted transcriptional activator comprising a catalytically dead CRISPR Cas protein linked to a transcription activation domain, and
b) at least one sgRNA molecule comprising nucleotide sequence that is complementary to the promoter of IL-15Ra, and
a second composition comprising:
c) a genome editing system of any one of claims 1-6 comprising a targeted transcriptional activator comprising a catalytically dead CRISPR Cas protein linked to a transcription activation domain, and
d) at least one sgRNA molecule comprising nucleotide sequence that is complementary to the promoter of IL-15.

19. The method of claim 17, wherein the disease or disorder is cancer or an infectious disease or disorder.

20. The method of claim 19, wherein the disease or disorder is HIV infection or AIDS.
